(19) United States
(12) Patent Application Publication
(10) Pub. No.: US 2018/0223213 A1
(43) Pub. Date: Aug. 9, 2018

(54) CHALCOGENIDE NANOOBJECTS AND USE THEREOF AS ADDITIVE

(71) Applicant: FUNDACIÓN CIDETEC, Donostia-San Sebastián (ES)

(72) Inventors: Ignacio GARCÍA ALONSO, Donostia-San Sebastián (ES); Sarah MONTES PÉREZ, Donostia-San Sebastián (ES); Ibon ODRIOZOLA, Donostia-San Sebastián (ES); Germán CABAÑERO, Donostia-San Sebastián (ES); Hans-Jürgen GRANDE, Donostia-San Sebastián (ES); Ramón TENA ZAERA, Donostia-San Sebastián (US)

(21) Appl. No.: 15/562,339

(22) PCT Filed: Mar. 31, 2016
(86) PCT No.: PCT/EP2016/057161

§ 371 Date: Sep. 27, 2017

(30) Foreign Application Priority Data
Apr. 1, 2015 (EP) 15382166.5

(51) Int. Cl. C10M 125/22; C07C 43/13; C07C 41/18; C07C 31/20; C07C 29/00; C07C 215/14; C07C 213/08; C08G 65/326; C07C 215/08; C07C 321/04; C07C 319/12; C08K 7/14; C08K 9/04; C08K 9/08; C08K 3/30; C10M 159/18; C01G 39/06

(52) U.S. Cl. CPC C10M 125/22; C01P 2004/62; C07C 41/18; C07C 31/205; C07C 29/00; C07C 215/14; C07C 213/08; C07C 31/20; C08G 65/326; C07C 215/08; C07C 321/04; C07C 319/12; C08K 7/14; C08K 9/04; C08K 9/08; C08K 3/30; C10M 159/18; C01G 39/06; C08K 2003/3009; C08K 2201/011; C10M 2201/066; C10M 2227/09; C10N 2220/082; C10N 2230/06; C01P 2002/88; C01P 2002/82; C01P 2004/03; C01P 2004/04; C01P 2004/13; C01P 2004/16; C01P 2004/32; C01P 2004/64; C07C 43/132

(57) ABSTRACT

The present invention provides a Molybdenum or Tungsten chalcogenide nanoobject having: (a) an object size comprised from 0.1 to 500 nm, and (b) from 1 to 99% by weight of molecules of formula (I) with respect to the total weight of the nanoobject, wherein: A is —OH; X is selected from: (C₁-C₂₀)alkyl optionally substituted with one or more radicals; a 2 to 20-member heteroalkyl optionally substituted with one or more radicals; and a homopolymer or copolymer comprising a polymeric chain; B is selected from: H, —OH, —NH₂, (C₁-C₄)alkyl, halogen, phenyl substituted with one or more halogen radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), — S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂), and —B(OR₁)(R₂); provided that when B is —H or (C₁-C₄) alkyl, then X is a homopolymer, a copolymer, or a 2 to 20-member heteroalkyl optionally substituted with one or more radicals as defined above.

The present invention also provides processes for the preparation of the nanoobjects, their use as additive for reducing the friction coefficient of a material, and compositions comprising thereof.

A-X-B (I)

This invention relates generally to lubrication and more specifically to lubricant compositions and their use.

### BACKGROUND ART

Metal chalcogenides exhibit technically useful, unique optical and electronic properties, which inspire the fabrication of their nanoparticles, nanosheets, nanotubes, and nanorods. Among them, an attractive vision is transition metal dichalcogenide of formula MX₂, wherein M is selected from Mo, W, Ti, Zr, Hf, V, Nb, and Ta, and X is selected from S, Se and Te, which constitutes a layered structure in analogy to graphite. The layered structure is constructed by unit X-M-X atomic trilayers which are connected by strongly covalent bonding and lattice layers. Due to this structure, a variety of applications for the metal chalcogenides can be found, including, for example, hydrodesulfurization catalysis, electrochemical intercalation, lubrication, hydrogen storage, elastic and coating materials, and Li batteries.

Lubricants are substances introduced to reduce friction between surfaces in mutual contact, which ultimately reduces the heat generated when the surfaces move. It may also have the function of transmitting forces, transporting foreign particles, or heating or cooling the surfaces. The property of reducing friction is known as lubricity. Typically the lubricant-to-surface friction is much less than surface-to-surface friction in a system without any lubrication. The addition of a lubricant in a particular material allows reducing the overall system friction. Reduced friction has the benefit of reducing heat generation and reduced formation of wear particles as well as improved efficiency and extension of life's material.

In addition to industrial applications, lubricants are used for many other purposes. Other uses include cooking (oils and fats in use in frying pans, in baking to prevent food sticking), bio-medical applications on humans (e.g. lubricants for artificial joints), ultrasound examination, and medical examinations, among others.

Nowadays, there is a trend among tribologists to investigate peculiar lubricious effects of nanostructured materials. Particular attention is given to nanocomposite lubricants. One of the most interesting challenges is to drastically decrease the friction coefficient between surfaces and to improve the antiwear action of nanosized particles of sandwich-like structures, for example, transition metal chalcogenides. Composite lubricants based on nanostructured metal dichalcogenides are promising materials due to their excellent tribological properties (low coefficient of friction, antiwear action). Previously, it was correlated the lowest coefficient of friction to the axial ratio which might not exceed 1.87. For 2H—MoS₂ and 2H—WS₂, the axial ratio is equal to 1.95 and 1.96, respectively. Under standard conditions, tungsten and molybdenum disulfides have almost the same coefficient of friction, but WS₂ reveals better thermal and oxidation stability than MoS₂. Recent interest of scientists has been focused on studying the superlubricity of nanomaterials. On the other hand, it has also been disclosed that WS₂ nanoobjects can be incorporated into a coating matrix to form nanocomposite coatings in order to affect its tribological properties.

One significant problem, however, with metal chalcogenide nanomaterials is the difficulty of controlling their dispersion inside an organic medium. Some works have addressed this problem and proposed to coat chalcogenide nanoobjects with non-polar structures (US 20050065044 and Parenago O. P. et al., “Synthesis and applications of inorganic nanoobjects as lubricant components—a review”, 2004, Journal of Nanoparticle Research, vol. 6, pages 273-284)

In spite of the efforts made, there is still the need of metal chalcogenides with appropriate friction coefficient and dispersibility.

### SUMMARY OF THE INVENTION

The present inventors have found that the functionalization of metal chalcogenide nanoobjects with a polar molecule reduces the friction coefficient in more than a 10% when compared with the corresponding non-functionalized metal chalcogenide nanoobject.

Due to this property, the nanoobjects of the invention are useful in reducing the friction of a particular material. Consequently, these functionalized metal chalcogenide of the invention can help in reducing heat generation, formation of wear particles, improving of the efficiency as well as prolonging the life of the product.

It has also been surprisingly found that the functionalization of such nanoobjects with such polar molecules, which confers to the resulting nanoobject a polar character, does not negatively affect to its dispersion in hydrophobic media, such a liquid hydrophobic lubricant oil. In other words, the polar nature of the functionalized nanoobject of the invention does not hinder the dispersion of the functionalized nanoobject in hydrophobic media. This makes the nanoobject of the invention useful as coefficient reduction additive, for example in lubricant oils.

Thus, the present invention provides in a first aspect a Molybdenum or Tungsten chalcogenide nanoobject having: (a) an object size comprised from 0.1 to 500 nm, and (b) from 1 to 99% by weight of molecules of formula (I) with respect to the total weight of the nanoobject

A-X-B (I)

wherein
A is —OH;
X is a biradical selected from the group consisting of: (C₁-C₂₀)alkyl; (C₁-C₂₀)alkyl substituted with one or more radicals independently selected from the group consisting of: (C₁-C₅)alkyl, —OH, halogen, phenyl, phenyl substituted with one or more (C₁-C₄)alkyl radicals, phenyl substituted with one or more halogen radicals, benzyl, benzyl substituted with one or more (C₁-C₄)alkyl radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), — S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂) and —B(OR₁)(R₂); a 2 to 20-member heteroalkyl; a 2 to 20-member heteroalkyl substituted with one or more radicals independently selected from the group consisting of: —OH, halogen, phenyl, phenyl substituted with one or more (C₁-C₄)alkyl radicals, phenyl substituted with one or more halogen radicals, benzyl, benzyl substituted with one or more (C₁-C₄)alkyl radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), — S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂) and —B(OR₁)(R₂); and a homopolymer or copolymer comprising a polymeric chain selected from the group consisting of: alkyd resin, epoxy resin, phenolic resin, polyvinyl halides, polyacetal, polyacrylics, polyalkylenes, polyalkenylenes, polyalkynylenes, polyamic acids, polyamides, polyamines, polyanhydrides, polyarylenealkylenes, polyarylenes, polyazomethines, polybenzimidazoles, polybenzothiazoles, polybenzyls, polycarbodiimides, polycarbonates, polycarbones, polycarboranes, polycarbosilanes, polycyanurates, polydienes, polyester-polyurethanes, polyesters, polyetheretherketones, polyether-polyurethanes, polyethers, polyhydrazides, polyimidazoles, polyimides, polyisocyanurates, polyketones, polyolefines, polyoxyalkylenes, polyoxyphenylenes, polyphenyls, polyphosphazenes, polypyrroles, polypyrrones, polyquinolines, polyquinoxalines, polysilanes, polysilazanes, polysiloxanes, polysilsesquioxanes, polysulfides, polysulfonamides, polysulfones, polythiazoles, polythiomethylenes, polythiophenylenes, polyureas, polyurethanes, polyvinyl acetals, polyvinyl butyrals, polyvinyl formals, polyvinyl alkanoates, vinyl polymers, and natural polymers;
B is a radical selected from the group consisting of: H, —OH, —NH₂, (C₁-C₄)alkyl, halogen, phenyl substituted with one or more halogen radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), —S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂) and —B(OR₁)(R₂);
provided that:
   when B is —H or (C₁-C₄)alkyl, then X is a 2 to 20-member heteroalkyl; a 2 to 20-member heteroalkyl substituted with one or more radicals, as defined above; or a homopolymer or copolymer, as defined above; and
   B is —H or (C₁-C₄)alkyl when X is a homopolymer, copolymer, a 2 to 20-member heteroalkyl or a 2 to 20-member heteroalkyl substituted as defined above; and
   when B is —NH₂, then X is a biradical selected from the group consisting of: (C₁-C₂₀)alkyl; (C₁-C₂₀)alkyl substituted with one or more (C₁-C₅)alkyl, —OH, halogen, phenyl, phenyl substituted with one or more (C₁-C₄)alkyl radicals, phenyl substituted with one or more halogen radicals, benzyl, benzyl substituted with one or more (C₁-C₄)alkyl radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), — S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂), and —B(OR₁)(R₂); a 2 to 20-member heteroalkyl; a 2 to 20-member heteroalkyl substituted with one or more —OH, halogen, phenyl, phenyl substituted with one or more (C₁-C₄)alkyl radicals, phenyl substituted with one or more halogen radicals, benzyl, benzyl substituted with one or more (C₁-C₄)alkyl radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), — S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂) and —B(OR₁)(R₂);
R₁, R₂, R₃, R₄, R₅, and R₆ are radicals independently selected from the group consisting of H, (C₁-C₂₀)alkyl, (C₅-C₁₂)aryl(C₁-C₂₀)alkyl and (C₅-C₁₂)aryl;
R₇ is halogen;
2 to 20-member heteroalkyl represents a known non-polymeric C-heteroalkyl radical consisting of from 2 to 20 members where at least one of the members is O, S, or NH, and the remaining members are selected from CH, C(═O), and CH₂; and
(C₅-C₁₂)aryl represents a ring system from 5 to 12 carbon atoms, the system comprising from 1 to 2 rings, where each one of the rings forming the ring system: is saturated, partially unsaturated, or aromatic; and is isolated, partially or totally fused.

The molecule (I) functionalizing the chalcogenide nanoobject of the present invention is of polar nature, either by the specific polar nature of A and B radicals, or by the specific polar nature of X when it is a homopolymer or copolymer from those listed in claim 1.

As it is illustrated below (see Table 2), the nature of A, B, and X determines the ability of the resulting nanoobject for reducing the friction coefficient of a lubricant oil. In this regard, in a comparative assay, a tribological test was performed with hexanedithiol and hexanediol, both molecules sharing the same “X” biradical (hexenyl) but differing in the meaning of A and B biradicals (—SH vs —OH). It was concluded with this comparative test that hexanedithiol was substantially less effective in reducing the friction coefficient of a lubricant oil. In particular, it was observed that hexanedithiol was about a 30% less efficient in reducing the friction coefficient than hexanediol.

Another aspect is a Molybdenum or Tungsten chalcogenide nanoobject having: (a) an object size comprised from 0.1 to 500 nm, and (b) from 1 to 99% by weight of molecules of formula (I) with respect to the total weight of the nanoobject

A-X-B (I)

wherein
A is a radical selected from the group consisting of —OH and —SH;
X is a biradical selected from the group consisting of: (C₁-C₂₀)alkyl; (C₁-C₂₀)alkyl substituted with one or more radicals independently selected from the group consisting of: (C₁-C₅)alkyl, —OH, halogen, phenyl, phenyl substituted with one or more (C₁-C₄)alkyl radicals, phenyl substituted with one or more halogen radicals, benzyl, benzyl substituted with one or more (C₁-C₄)alkyl radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), — S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂) and —B(OR₁)(R₂); a 2 to 20-member heteroalkyl; a 2 to 20-member heteroalkyl substituted with one or more radicals independently selected from the group consisting of: —OH, halogen, phenyl, phenyl substituted with one or more (C₁-C₄)alkyl radicals, phenyl substituted with one or more halogen radicals, benzyl, benzyl substituted with one or more (C₁-C₄)alkyl radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), — S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂) and —B(OR₁)(R₂); and a homopolymer or copolymer comprising a polymeric chain selected from the group consisting of: alkyd resin, epoxy resin, phenolic resin, polyvinyl halides, polyacetal, polyacrylics, polyalkylenes, polyalkenylenes, polyalkynylenes, polyamic acids, polyamides, polyamines, polyanhydrides, polyarylenealkylenes, polyarylenes, polyazomethines, polybenzimidazoles, polybenzothiazoles, polybenzyls, polycarbodiimides, polycarbonates, polycarbones, polycarboranes, polycarbosilanes, polycyanurates, polydienes, polyester-polyurethanes, polyesters, polyetheretherketones, polyether-polyurethanes, polyethers, polyhydrazides, polyimidazoles, polyimides, polyisocyanurates, polyketones, polyolefines, polyoxyalkylenes, polyoxyphenylenes, polyphenyls, polyphosphazenes, polypyrroles, polypyrrones, polyquinolines, polyquinoxalines, polysilanes, polysilazanes, polysiloxanes, polysilsesquioxanes, polysulfides, polysulfonamides, polysulfones, polythiazoles, polythiomethylenes, polythiophenylenes, polyureas, polyurethanes, polyvinyl acetals, polyvinyl butyrals, polyvinyl formals, polyvinyl alkanoates, vinyl polymers, and natural polymers; B is a radical selected from the group consisting of: H, —OH, halogen, phenyl substituted with one or more halogen radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), — S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂) and —B(OR₁)(R₂);
R₁, R₂, R₃, R₄, R₅, and R₆ are radicals independently selected from the group consisting of H, (C₁-C₂₀)alkyl, (C₅-C₁₂)aryl(C₁-C₂₀)alkyl and (C₅-C₁₂)aryl;
R₇ is halogen;
2 to 20-member heteroalkyl represents a known non-polymeric C-heteroalkyl radical consisting of from 2 to 20 members where at least one of the members is O, S, or NH, and the remaining members are selected from CH, C(═O), and CH₂; and
(C₅-C₁₂)aryl represents a ring system from 5 to 12 carbon atoms, the system comprising from 1 to 2 rings, where each one of the rings forming the ring system: is saturated, partially unsaturated, or aromatic; and is isolated, partially or totally fused.

The present invention provides, in a second aspect, a process for preparing a nanoobject as defined in the first aspect of the invention, comprising the step of reacting a Molybdenum or Tungsten source with a source of S, Se or Te and molecules of formula (I) as defined above.

The nanoobject of the present invention may be also defined by its preparation process. Thus, it is also part of the invention, as a third aspect, a nanobject obtainable by the process defined in the second aspect of the invention.

As it has been highlighted above, the nanoobjects of the present invention show reduced coefficient friction, which make them useful for reducing the friction of a material.

Thus, in a fourth aspect the present invention provides the use of a nanoobject as defined in the first or third aspect for reducing the friction coefficient of a material.

With the use of the fourth aspect of the invention, it is encompassed the use of the nanoobject of the invention as lubricant per se or as additive to be added to a material, such as to a lubricant oil.

In a fifth aspect, the present invention provides an article comprising the nanoobject as defined in the first and third aspects of the invention.

As shown below, the inclusion of nanoobjects with molecules of formula (I) in a lubricant oil allows reducing the friction coefficient of the oil in at least about a 40% (see Table 2 below).

Finally, the present invention also provides lubricant oil compositions and nanocomposites comprising the nanoobjects of the first and third aspects of the invention.

Tables 4 and 5 below show that nanoobjects functionalized with molecules of formula (I) have a huge influence in the reduction of the wear of glass fibre reinforced polyamide (PA) or polypropylene (PP) polymeric matrices. In fact, as it is shown below the reduction is at least of about 16%.

### DETAILED DESCRIPTION OF THE INVENTION

In the present invention the term “nanoobject” refers to a primary particle (non-agglomerated single particle) with one, two or three external dimensions in the nanoscale, as already recognized by International Organization for Standardization in the document with the reference number ISO/TS 27687:2008(E). Illustrative non-limitative examples of nanoobjects are: nanoparticles, which are nanoobjects with all three external dimensions in the nanoscale (if the lengths of the longest to the shortest axes of the nanoobject differ significantly, typically by more than three times, the terms nanofibre or nanoplate are intended to be used instead of the term nanoparticle); nanosheets (or nanoplates or nanolayers), which are nanoobjects with one external dimension in the nanoscale and the two other external dimensions significantly larger, wherein the smallest external dimension is the thickness of the nanosheets, the two significantly larger dimensions are considered to differ from the nanoscale dimension by more than three times, and the larger external dimensions are not necessarily in the nanoscale; nanofibres, which are nanoobjects with two similar external dimensions in the nanoscale and the third dimension significantly larger, wherein the nanofibres can be flexible or rigid and the two similar external dimensions are considered to differ in size by less than three times and the significantly larger external dimension is considered to differ from the other two by more than three times, and the largest external dimension is not necessarily in the nanoscale; nanotubes, which are hollow nanofibre; nanorods, which are solid nanofibre; nanowires, which are electrically conducting or semi-conducting nanofibres; and quantum dots, which are crystalline nanoparticles exhibiting size-dependent properties due to quantum confinement effects on the electronic states.

The term “object size” when referred to the nanoobject of the invention refers to a characteristic physical dimension of the primary particle. For example, in the case of a spherical nanoobject, the “object size” corresponds to the diameter of the nanoobject. In the case of a rod-shaped nanoobject with a circular cross-section, as it is the case of a nanofibre (either as such or in the form of a nanowire or nanotube), the “object size” of the nanoobject corresponds to the diameter of the cross-section of the nanoobject. In the case of a box-shaped nanoobject, such as a nanosheet, nanocube, a nanobox, or a nanocage, the size of the nanoobject corresponds to the thickness. When referring to a set of nanoobjects being of a particular size, it is contemplated that the set of nanoobjects can have a distribution of sizes around the specified size.

The size of the nanoobjects of the invention can be determined using well-known techniques in the state of the art such as Transmission Electron Microscopy (TEM). Images were chosen to be as representative of bulk sample as possible. TEM observations were performed on a JEOL 2010 F operating with 200 KV accelerating voltage equipped with Energy Dispersive Spectroscopy (EDS). The measured dimension was chosen depending on the morphology of the nanoobject as described above.

In the present invention, the term “chalcogenide” means a chemical compound consisting of at least one chalcogen anion and at least one more electropositive element. In one embodiment, the chalcogenide is a sulfide, selenide or telluride.

In the present invention, the term “polymeric chain” means a molecule of high relative molecular mass, the structure of which essentially comprises the multiple repetition of units derived, actually or conceptually, from molecules of low relative molecular mass.

In the present invention the term “natural polymers” can be defined as naturally occurring polymers which are produced in living organisms. The most important naturally occurring polymers are proteins, polysaccharides (e.g. cellulose, starch, cotton), nucleic acids (e.g. DNA, RNA) and natural rubber.

According to the present invention a ring system formed by “isolated” rings means that the ring system is formed by two rings and said rings are bound via a bond from the atom of one ring to the atom of the other ring. The term “isolated” also embraces the embodiment in which the ring system has only one ring. Illustrative non-limitative examples of known ring systems consisting of one ring are those derived from: cyclopropyl, cyclobutyl, cyclopentyl, cyclohexyl, cycloheptyl, cyclopropenyl, cyclobutenyl, cyclopentenyl, phenyl, biphenylyl, and cycloheptenyl.

According to the present invention when the ring system has “totally fused” rings, it means that the ring system is formed by two rings in which two or more atoms are common to two adjoining rings. Illustrative non-limitative examples are 1,2,3,4-tetrahydronaphthyl, and 1-naphthyl, 2-naphthyl. In the present invention, the term “(%) by weight” refers to the percentage of each ingredient of the nanoobject or composition in relation to the total weight. As it is explained in detail below, the % by weight of molecules of formula (I) in relation of the total weight of the nanoobject has been determined by Thermal gravimetric analysis (TGA).

In one embodiment of the first aspect of the invention the metal chalcogenide is a Molybdenum chalcogenide of sulfide, selenide or telleride. In another embodiment of the first aspect of the invention the metal chalcogenide is MoS₂.

Molybdenum disulfide is a solid lubricant relating to the class of inorganic lubricants with lamellar structure. The crystal lattice of molybdenum disulfide is similar to that of Graphite. It consists of hexagonal molybdenum planes sandwiched between two hexagonal sulfur planes. The atoms in the planes are strongly covalently bonded to each other. The planes are bonded by weak Van der Waals forces. The layered structure allows sliding movement of the parallel plates. Weak bonding between the planes provides low shear strength in the direction of the sliding movement but high compression strength in the direction perpendicular to the sliding movement. Friction forces cause the particles of molybdenum disulfide to orient in the direction, in which the hexagonal layers are parallel to the sliding movement. The anisotropy of the mechanical properties imparts the combination of low coefficient of friction and high carrying load capacity to molybdenum disulfide. The sulfur layers of molybdenum disulfide have an affinity for tenacious adherence to the metal substrate atoms therefore a strong lubrication film is formed on the substrate surface. The lubrication film provides good wear resistance and seizure resistance (compatibility).

Surprisingly it has been found that when the MoS₂ is functionalized with molecules of formula (I), there is a reduction in the friction coefficient up to about 16% when compared with non-functionalized MoS₂ nanoobjects.

In one embodiment of the first aspect of the invention, the nanoobject comprises from 15 to 99% by weight of molecules of formula (I) with respect to the total weight of the nanoobject. In one embodiment of the first aspect of the invention, the nanoobject comprises from 30 to 99% by weight of molecules of formula (I) with respect to the total weight of the nanoobject. In another embodiment, the nanoobject comprises from 40 to 99% by weight of molecules of formula (I) with respect to the total weight of the nanoobject. In another embodiment, the nanoobject comprises from 40 to 95% by weight of molecules of formula (I) with respect to the total weight of the nanoobject.

In another embodiment, X is a homopolymer or copolymer comprising a polymeric chain selected from the group consisting of: alkyd resin, epoxy resin, phenolic resin, polyvinyl halides, polyacetal, polyacrylics, polyalkylenes, polyalkenylenes, polyalkynylenes, polyamides, polyamines, polyanhydrides, polycarbonates, polyester-polyurethanes, polyesters, polyetheretherketones, polyether-polyurethanes, polyethers, polyimidazoles, polyimides, polyisocyanurates, polyketones, polyolefines, polyoxyalkylenes, polyoxyphenylenes, polypyrroles, polysiloxanes, polysulfides, polysulfonamides, polysulfones, polythiazoles, polythiomethylenes, polythiophenylenes, polyureas, polyurethanes, polyvinyl acetals, polyvinyl butyrals, polyvinyl formals polyvinyl alkanoates, vinyl polymers, and natural polymers. In another embodiment, X is a homopolymer or copolymer comprising a polymeric chain selected from the group consisting of: epoxy resin, phenolic resin, polyvinyl halides, polyacetal, polyacrylics, polyamides, polyamines, polycarbonates, polyester-polyurethanes, polyesters, polyether-polyurethanes, polyethers, polyimides, polyketones, polyolefines, polyoxyalkylenes, polyoxyphenylenes, polysiloxanes, polysulfides, polysulfones, polythiomethylenes, polyureas, polyurethanes, polyvinyl acetals, and polyvinyl alkanoates, and natural polymers. In still another embodiment X is a polyether. Illustrative non-limitative examples of polyethers are: polyoxymethylene (POM), polyacetal, polyethylene oxide (PEO), polypropylene oxide (PPO), polytetrahydrofuran (PTHF). In still another embodiment, X is a polyethylene oxide. In still another embodiment, X is a polyether, A is —OH, and B is selected from —H, and (C₁-C₄) alkyl. In still another embodiment, X is a polyether and A and B are —OH.

In another embodiment of the first aspect of the invention the molecule of formula (I) is one wherein X is a biradical selected from the group consisting of: (C₁-C₁₀)alkyl; (C₁-C₁₀)alkyl substituted with one or more radicals as defined in the first aspect of the invention; a 2 to 10-member heteroalkyl; a 2 to 10-member heteroalkyl substituted with one or more radicals as defined in the first aspect of the invention; and a homopolymer or copolymer as defined in the first aspect of the invention. In another embodiment, the compound of formula (I) is one wherein X is a biradical selected from the group consisting of: (C₁-C₁₀)alkyl; a 2 to 10-member heteroalkyl; and a 2 to 10-member heteroalkyl substituted with one or more (C₁-C₅)alkyl radicals. In another embodiment X is a biradical selected from the group consisting of: (C₁-C₁₀)alkyl; and 2 to 10-member heteroalkyl as defined in the first aspect of the invention above. In another embodiment X is a 2 to 10-member heteroalkyl having from 2 to 10 members, being at least one of the members selected from O, S, and NH, and the remaining members are CH₂ members. In another embodiment X is selected from (C₁-C₁₀)alkyl, and a 2 to 10-member heteroalkyl having from 2 to 10 members, being at least one of the members selected from O, and NH, and the remaining members are CH₂ members. In another embodiment X is selected from (C₁-C₁₀)alkyl, and a 2 to 10-member heteroalkyl having from 2 to 10 members, being one or two of the members independently selected from O, and NH, and the remaining members are CH₂ members. In another embodiment X is selected from (C₁-C₁₀)alkyl, and a 2 to 10-member heteroalkyl having from 2 to 10 members, being one or two of them O members and the remaining being CH₂ members. In another embodiment X is selected from (C₁-C₁₀)alkyl, and a 2 to 10-member heteroalkyl having from 2 to 10 members, being one of the members NH, and the remaining being CH₂ members.

In another embodiment of the first aspect of the invention the molecule of formula (I) is one wherein X is a biradical selected from the group consisting of: (C₁-C₆)alkyl; (C₁-C₆)alkyl substituted with one or more radicals as defined in the first aspect of the invention; a 2 to 6-member heteroalkyl; a 2 to 6-member heteroalkyl substituted with one or more radicals as defined in the first aspect of the invention; and a homopolymer or copolymer as defined in the first aspect of the invention. In another embodiment, the compound of formula (I) is one wherein X is a biradical selected from the group consisting of: (C₁-C₆)alkyl; a 2 to 6-member heteroalkyl; and a 2 to 6-member heteroalkyl substituted with one or more (C₁-C₅)alkyl radicals. In another embodiment X is a biradical selected from the group consisting of: (C₁-C₆)alkyl; and 2 to 6-member heteroalkyl as defined in the first aspect of the invention. In another embodiment X is a 2 to 6-member heteroalkyl having from 2 to 6 members, being at least one of the members selected from O, S, and NH, and the remaining members are CH₂ members. In another embodiment X is selected from (C₁-C₆)alkyl, and a 2 to 6-member heteroalkyl having from 2 to 6 members, being at least one of the members selected from O, and NH, and the remaining members are CH₂ members. In another embodiment X is selected from (C₁-C₆)alkyl, and a 2 to 6-member heteroalkyl having from 2 to 6 members, being one or two of the members independently selected from O, and NH, and the remaining members being CH₂ members. In another embodiment X is selected from (C₁-C₆)alkyl, and a 2 to 6-member heteroalkyl having from 2 to 6 members, being one or two of them O member(s), and the remaining being CH₂ members. In another embodiment X is selected from (C₁-C₆)alkyl, and a 2 to 6-member heteroalkyl having from 2 to 6 members, being one of them being a NH member, and the remaining members being CH₂ members.

In another embodiment of the first aspect of the invention, the nanoobject comprises from 1 to 99%, from 20 to 80% or from 30 to 70% by weight of molecules of formula (I) with respect to the total weight of the nanoobject when X is a biradical selected from the group consisting of: (C₁-C₂₀)alkyl; and 2 to 20-member heteroalkyl as defined above. In another embodiment of the first aspect of the invention, the nanoobject comprises from 1 to 99%, from 20 to 80% or from 30 to 70% of molecules of formula (I) with respect to the total weight of the nanoobject when X is (C₁-C₁₀)alkyl, or a 2 to 10-member heteroalkyl having from 2 to 10 members, being at least one of the members selected from O, S, and NH, and the remaining being CH₂ members. In another embodiment of the first aspect of the invention, the nanoobject comprises from 1 to 99%, from 20 to 80% or from 30 to 70% of molecules of formula (I) with respect to the total weight of the nanoobject when X is (C₁-C₆)alkyl, or a 2 to 6-member heteroalkyl having from 2 to 6 members, being at least one of the members selected from O, S, and NH, and the remaining being CH₂ members. In another embodiment of the first aspect of the invention, the nanoobject comprises from 1 to 99%, from 20 to 80% or from 30 to 70% of molecules of formula (I) with respect to the total weight of the nanoobject when X is (C₁-C₁₀)alkyl or a 2 to 10-member heteroalkyl having from 2 to 10 members, being at least one of the members selected from O, and NH, and the remaining being CH₂ members. In another embodiment of the first aspect of the invention, the nanoobject comprises from 1 to 99%, from 20 to 80% or from 30 to 70% of molecules of formula (I) with respect to the total weight of the nanoobject when X is (C₁-C₆)alkyl or a 2 to 6-member heteroalkyl having from 2 to 6 members, being at least one of the members selected from O, and NH, and the remaining being CH₂ members. In another embodiment of the first aspect of the invention, the nanoobject comprises from 1 to 99%, from 20 to 80% or from 30 to 70% by weight of molecules of formula (I) with respect to the total weight of the nanoobject when X is selected from (C₁-C₁₀)alkyl, and a 2 to 10-member heteroalkyl having from 2 to 10 members, being one or two of the members independently selected from O, and NH, and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject when X is selected from (C₁-C₆)alkyl, and a 2 to 6-member heteroalkyl having from 2 to 6 members, being one or two of the members independently selected from O, and NH, and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject when X is selected from (C₁-C₁₀)alkyl, and a 2 to 10-member heteroalkyl having from 2 to 10 members, being one or two of them being O member(s), and the remaining being CH₂ members. In another embodiment of the first aspect of the invention, the nanoobject comprises from 1 to 99%, from 20 to 80% or from 30 to 70% by weight of molecules of formula (I) with respect to the total weight of the nanoobject when X is selected from (C₁-C₆)alkyl, and a 2 to 6-member heteroalkyl having from 2 to 6 members, being one or two of them being O member(s), and the remaining being CH₂ members. In another embodiment of the first aspect of the invention, the nanoobject comprises from 1 to 99%, from 20 to 80% or from 30 to 70% by weight of molecules of formula (I) with respect to the total weight of the nanoobject when X is selected from (C₁-C₁₀)alkyl, and a 2 to 10-member heteroalkyl having from 2 to 10 members, being one of them a NH member, and the remaining being CH₂ members. In another embodiment of the first aspect of the invention, the nanoobject comprises from 1 to 99%, from 20 to 80% or from 30 to 70% by weight of molecules of formula (I) with respect to the total weight of the nanoobject when X is selected from (C₁-C₆)alkyl, and a 2 to 6-member heteroalkyl having from 2 to 6 members, being one of them a NH member, and the remaining being CH₂ members.

In the present invention, the expression “have(has) from” has the same meaning as “comprise(s) from”.

In another embodiment of the first aspect of the invention, the nanoobject comprises from 1 to 99%, from 15 to 99%, from 30 to 99% or from 90 to 99% by weight of molecules of formula (I) with respect to the total weight of the nanoobject, when X is a homopolymer or copolymer, as defined above. In another embodiment of the first aspect of the invention, the nanoobject comprises from 1 to 99%, from 15 to 99%, from 30 to 99% or from 90 to 99% by weight of molecules of formula (I) with respect to the total weight of the nanoobject, when X is a homopolymer or copolymer comprising a polyether polymeric chain. In another embodiment of the first aspect of the invention, the nanoobject comprises from 1 to 99%, from 15 to 99%, from 30 to 99% or from 90 to 99% by weight of molecules of formula (I) with respect to the total weight of the nannoobject when X is a homopolymer or copolymer comprising a polyethylene oxide polymeric chain.

In another embodiment of the first aspect of the invention, B is a radical selected from the group consisting of: H, —NH₂, (C₁-C₄)alkyl, —OH, halogen, phenyl substituted with one or more halogen radicals, benzyl substituted with one or more halogen radicals, —C(═O)R₃, —OC(═O)(O)R₃, —C(═O)(O⁻), —C(═O)(O)R₃, —OR₃, —CH(OR₃)(OH), —C(OR₃)(OH)(R₄), —CH(OR₃)(OR₄), NR₁R₂, N⁺R₁R₂R₃, —C(═NR₁)(H), —C(═O)(NR₁R₂), —N(C(═O)(R₁))(C(═O)(R₂))(R₃), —O(CN), —NC(═O), —ONO₂, —CN, —NC, —ON(═O), —NO₂, —NO, —C₅H₄N (pyridyl), —SR₁, —SSR₃, —S(═O)(R₁), —S(═O)(═O)(R₁), —S(═O)(OH), —S(═O)(═O)(OH), —SCN, —NCS, —C(═S)(H), —P(═O)(OH)₂, —OP(═O)(OH)₂, —B(OH), —B(OR₁)(OR₂), and —B(OH)(R₁). In another embodiment, B is a radical selected from the group consisting of: H, —NH₂, (C₁-C₄)alkyl, OH, halogen, phenyl substituted with one or more halogen radicals, —C(═O)(O⁻), —C(═O)(O)R₃, —OR₃, NR₁R₂, N⁺R₁R₂R₃, —C(═O)(NR₁R₂), —ONO₂—CN (nitrile), —NC, —NO₂, —NO, —C₅H₄N, —SR₁, —S(═O)(═O)(R₁), —S(═O)(═O)(OH), —OP(═O)(OH)₂, —B(OH) and —B(OH)(R₁). In still another embodiment B is H, —NH₂, (C₁-C₄)alkyl, or OH.

In another embodiment of the first aspect of the invention, B is a radical selected from the group consisting of: H, —OH, halogen, phenyl substituted with one or more halogen radicals, benzyl substituted with one or more halogen radicals, —C(═O)R₃, —OC(═O)(O)R₃, —C(═O)(O⁻), —C(═O)(O)R₃, —OR₃, —CH(OR₃)(OH), —C(OR₃)(OH)(R₄), —CH(OR₃)(OR₄), NR₁R₂, N⁺R₁R₂R₃, —C(═NR₁)(H), —C(═O)(NR₁R₂), —N(C(═O)(R₁))(C(═O)(R₂))(R₃), —O(CN), —NC(═O), —ONO₂, —CN, —NC, —ON(═O), —NO₂, —NO, —C₅H₄N (pyridyl), —SR₁, —SSR₃, —S(═O)(R₁), —S(═O)(═O)(R₁), —S(═O)(OH), —S(═O)(═O)(OH), —SCN, —NCS, —C(═S)(H), —P(═O)(OH)₂, —OP(═O)(OH)₂, —B(OH), —B(OR₁)(OR₂), and —B(OH)(R₁). In another embodiment, B is a radical selected from the group consisting of: H, OH, halogen, phenyl substituted with one or more halogen radicals, —C(═O)(O⁻), —C(═O)(O)R₃, —OR₃, NR₁R₂, N⁺R₁R₂R₃, —C(═O)(NR₁R₂), —ONO₂—CN (nitrile), —NC, —NO₂, —NO, —C₅H₄N, —SR₁, —S(═O)(═O)(R₁), —S(═O)(═O)(OH), —OP(═O)(OH)₂, —B(OH) and —B(OH)(R₁). In still another embodiment B is H or OH.

In one embodiment of the first aspect of the invention, the molecule of formula (I) is one wherein R₁, R₂, R₃, R₄, R₅, and R₆ are radicals independently selected from the group consisting of H, (C₁-C₁₀)alkyl, (C₅-C₁₂)aryl(C₁-C₁₀)alkyl and (C₅-C₁₂)aryl. In another embodiment of the first aspect of the invention, the molecule of formula (I) is one wherein R₁, R₂, R₃, R₄, R₅, and R₆ are radicals independently selected from the group consisting of H, (C₁-C₃)alkyl, (C₅-C₁₂)aryl(C₁-C₃)alkyl and (C₅-C₁₂)aryl.

In another embodiment of the first aspect of the invention, the molecule of formula (I) is one where A represents —OH, and B and X are as defined in any of the above embodiments.

In another embodiment of the first aspect of the invention, the molecule of formula (I) is one wherein A represents —OH, B is H, OH, —NH₂, or (C₁-C₄)alkyl, and X is as defined in any of the above embodiments. In another embodiment of the first aspect of the invention, the molecule of formula (I) is one wherein B is —OH or H, and A and X are as defined in any of the above embodiments. In another embodiment of the first aspect of the invention, the molecule of formula (I) is one wherein A represents —OH, B is —OH or H, and X is as defined in any of the above embodiments.

In another embodiment, the molecule of formula (I) is one selected from the group consisting of propylene glycol, ethylene glycol, diethylene glycol, polyethylene glycol, diethanolamine, 1,6-hexanediol, polyethyleneglycolmonomethyl ether, and 6-amino-1-hexanol.

In another embodiment, the molecule of formula (I) is one selected from the group consisting of propylene glycol, ethylene glycol, diethylene glycol, polyethylene glycol, diethanolamine, and 1,6-hexanediol.

In another embodiment of the first aspect of the invention, the nanoobject comprises from 1 to 99%, from 15 to 99%, from 20 to 80% or from 30 to 70% by weight of molecules of formula (I) with respect to the total weight of the nanoobject, when: A is —OH, B is —NH₂, (C₁-C₄)alkyl, —OH or H, and X is a biradical selected from the group consisting of: (C₁-C₂₀)alkyl; and 2 to 20-member heteroalkyl as defined above. In another embodiment of the first aspect of the invention, the nanoobject comprises from 1 to 99%, from 15 to 99%, from 20 to 80% or from 30 to 70% by weight of molecules of formula (I) with respect to the total weight of the nanoobject, when: A is —OH, B is —NH₂, (C₁-C₄)alkyl, —OH or H, and X is selected from (C₁-C₁₀)alkyl and a 2 to 10-member heteroalkyl having from 2 to 10 members, being at least one of the members selected from O, S, and NH, and the remaining members are CH₂ members. In another embodiment of the first aspect of the invention, the nanoobject comprises from 1 to 99%, from 15 to 99%, from 20 to 80% or from 30 to 70% by weight of molecules of formula (I) with respect to the total weight of the nanoobject, when: A is —OH, B is —NH₂, (C₁-C₄)alkyl, —OH or H, and X is selected from (C₁-C₆)alkyl and a 2 to 6-member heteroalkyl having from 2 to 6 members, being at least one of the members selected from O, S, and NH, and the remaining members are CH₂ members. In another embodiment the nanoobject comprises from 1 to 99%, from 15 to 99%, from 20 to 80% or from 30 to 70% by weight of molecules of formula (I) with respect to the total weight of the nanoobject, when: A is —OH, B is —NH₂, (C₁-C₄)alkyl, —OH or H, and X is selected from (C₁-C₁₀)alkyl or a 2 to 10-member heteroalkyl having from 2 to members, being at least one of the members selected from O, and NH, and the remaining members are CH₂ members. In another embodiment the nanoobject comprises from 1 to 99%, from 15 to 99%, from 20 to 80% or from 30 to 70% by weight of molecules of formula (I) with respect to the total weight of the nanoobject, when: A is —OH, B is —NH₂, (C₁-C₄)alkyl, —OH or H, and X is selected from (C₁-C₆)alkyl or a 2 to 6-member heteroalkyl having from 2 to 6 members, being at least one of the members selected from O, and NH, and the remaining members are CH₂ members. In another embodiment of the first aspect of the invention, the nanoobject comprises from 1 to 99%, from 15 to 99%, from 20 to 80% or from 30 to 70% by weight of molecules of formula (I) with respect to the total weight of the nanoobject, when: A is —OH, B is —NH₂, (C₁-C₄)alkyl, —OH or H, and X is a biradical selected from (C₁-C₁₀)alkyl, and a 2 to 10-member heteroalkyl having from 2 to 10 members, being one or two of the members independently selected from O, and NH, and the remaining members being CH₂ members. In another embodiment of the first aspect of the invention, the nanoobject comprises from 1 to 99%, from 15 to 99%, from 20 to 80% or from 30 to 70% by weight of molecules of formula (I) with respect to the total weight of the nanoobject, when: A is —OH, B is —NH₂, (C₁-C₄)alkyl, OH, or H, and X is a biradical selected from (C₁-C₆)alkyl, and a 2 to 6-member heteroalkyl having from 2 to 6 members, being one or two of the members independently selected from O, and NH, and the remaining members being CH₂ members. In another embodiment of the first aspect of the invention, the nanoobject comprises from 1 to 99%, from 15 to 99%, from 20 to 80% or from 30 to 70% by weight of molecules of formula (I) with respect to the total weight of the nanoobject, when: A is —OH, B is —NH₂, (C₁-C₄)alkyl, OH, H, and X is a biradical selected from (C₁-C₁₀)alkyl, and a 2 to 10-member heteroalkyl having from 2 to 10 members, being one or two of them O member(s), and the remaining members being CH₂ members. In another embodiment of the first aspect of the invention, the nanoobject comprises from 1 to 99%, from 15 to 99%, from 20 to 80% or from 30 to 70% by weight of molecules of formula (I) with respect to the total weight of the nanoobject, when: A is —OH, B is —NH₂, (C₁-C₄)alkyl, —OH or H, and X is a biradical X selected from (C₁-C₆)alkyl, and a 2 to 6-member heteroalkyl having from 2 to 6 members, being one or two of them O member(s), and the remaining members being CH₂ members. In another embodiment of the first aspect of the invention, the nanoobject comprises from 1 to 99%, from 15 to 99%, from 20 to 80% or from 30 to 70% by weight of molecules of formula (I) with respect to the total weight of the nanoobject, when: A is —OH, B is —NH₂, (C₁-C₄)alkyl, OH, or H, and X is a biradical selected from (C₁-C₁₀)alkyl, and a 2 to 10-member heteroalkyl having from 2 to 10 members, being one of them being a NH member, and the remaining members being CH₂ members. In another embodiment of the first aspect of the invention, the nanoobject comprises from 1 to 99%, from 15 to 99%, from 20 to 80% or from 30 to 70% by weight of molecules of formula (I) with respect to the total weight of the nanoobject, when: A is —OH, B is —NH₂, (C₁-C₄)alkyl, —OH or H, and X is a biradical selected from (C₁-C₆)alkyl, and a 2 to 6-member heteroalkyl having from 2 to 6 members, being one of them being a NH member, and the remaining members being CH₂ members.

In another embodiment of the first aspect of the invention, the nanoobject comprises from 1 to 99%, from 20 to 80% or from 30 to 70% by weight of molecules of formula (I) with respect to the total weight of the nanoobject, when: A is —OH or —SH, B is —OH or H, and X is a biradical selected from the group consisting of: (C₁-C₂₀)alkyl; and 2 to 20-member heteroalkyl as defined above. In another embodiment of the first aspect of the invention, the nanoobject comprises from 1 to 99%, from 20 to 80% or from 30 to 70% by weight of molecules of formula (I) with respect to the total weight of the nanoobject, when: A is —OH or —SH, B is —OH or H, and X is selected from (C₁-C₁₀)alkyl and a 2 to 10-member heteroalkyl having from 2 to 10 members, being at least one of the members selected from O, S, and NH, and the remaining members are CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH or —SH, B is —OH or H, and X is selected from (C₁-C₆)alkyl and a 2 to 6-member heteroalkyl having from 2 to 6 members, being at least one of the members selected from O, S, and NH, and the remaining members are CH₂ members. In another embodiment the nanoobject comprises from 1 to 99%, from 20 to 80% or from 30 to 70% by weight of molecules of formula (I) with respect to the total weight of the nanoobject, when: A is —OH or —SH, B is —OH or H, and X is selected from (C₁-C₁₀)alkyl or a 2 to 10-member heteroalkyl having from 2 to 10 members, being at least one of the members selected from O, and NH, and the remaining members are CH₂ members. In another embodiment the nanoobject comprises from 1 to 99%, from 20 to 80% or from 30 to 70% by weight of molecules of formula (I) with respect to the total weight of the nanoobject, when: A is —OH or —SH, B is —OH or H, and X is selected from (C₁-C₆)alkyl or a 2 to 6-member heteroalkyl having from 2 to 6 members, being at least one of the members selected from O, and NH, and the remaining members are CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH or —SH, B is —OH or H, and X is a biradical selected from (C₁-C₁₀)alkyl, and a 2 to 10-member heteroalkyl having from 2 to 10 members, being one or two of the members independently selected from O, and NH, and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH or —SH, B is —OH or H, and X is a biradical selected from (C₁-C₆)alkyl, and a 2 to 6-member heteroalkyl having from 2 to 6 members, being one or two of the members independently selected from O, and NH, and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH or —SH, B is —OH or H, and X is a biradical X selected from (C₁-C₆)alkyl, and a 2 to 10-member heteroalkyl having from 2 to 10 members, being one or two of them O member(s), and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH or —SH, B is —OH or H, and X is a biradical X selected from (C₁-C₆)alkyl, and a 2 to 6-member heteroalkyl having from 2 to 6 members, being one or two of them O member(s), and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH or —SH, B is —OH or H, and X is a biradical selected from (C₁-C₁₀)alkyl, and a 2 to 10-member heteroalkyl having from 2 to 10 members, being one of them being a NH member, and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH or —SH, B is —OH or H, and X is a biradical selected from (C₁-C₆)alkyl, and a 2 to 6-member heteroalkyl having from 2 to 6 members, being one of them being a NH member, and the remaining members being CH₂ members.

In another embodiment of the first aspect of the invention, the nanoobject comprises from 1 to 99%, from 15 to 99%, from 30 to 99% or from 90 to 99% by weight of molecules of formula (I) with respect to the total weight of the nanoobject when: A is —OH, B is (C₁-C₄)alkyl, —OH or H, and X is a homopolymer or copolymer as defined in the first aspect of the invention. In another embodiment, the nanoobject comprises from 1 to 99%, from 15 to 99%, from 30 to 99% or from 90 to 99% by weight of molecules of formula (I) with respect to the total weight of the nanoobject, when: A is —OH, B is (C₁-C₄)alkyl, —OH or H, and X is copolymer or homopolymer comprising a polyether chain. In another embodiment of the first aspect of the invention, the nanoobject comprises from 1 to 99%, from 15 to 99%, from 30 to 99% or from 90 to 99% by weight of molecules of formula (I) with respect to the total weight of the nanoobject when: A is —OH, B is (C₁-C₄)alkyl, —OH or H, and X is a homopolymer or copolymer comprising a polyethylene oxide chain.

In another embodiment of the first aspect of the invention, the nanoobject comprises from 1 to 99%, from 30 to 99% or from 90 to 99% by weight of molecules of formula (I) with respect to the total weight of the nanoobject when: A is —OH or —SH, B is —OH or H, and X is a homopolymer or copolymer as defined in the first aspect of the invention. In another embodiment, the nanoobject comprises from 1 to 99%, from 30 to 99% or from 90 to 99% by weight of molecules of formula (I) with respect to the total weight of the nanoobject, when: A is —OH or —SH, B is —OH or H, and X is copolymer or homopolymer comprising a polyether chain. In another embodiment of the first aspect of the invention, the nanoobject comprises from 1 to 99%, from 30 to 99% or from 90 to 99% by weight of molecules of formula (I) with respect to the total weight of the nanoobject when: A is —OH or —SH, B is —OH or H, and X is a homopolymer or copolymer comprising a polyethylene oxide chain.

In another embodiment of the first aspect of the invention, the nanoobject comprises from 1 to 99%, from 20 to 80% or from 30 to 70% by weight of molecules of formula (I) with respect to the total weight of the nanoobject, when: A is —OH, B is —OH or H, and X is a biradical selected from the group consisting of: (C₁-C₂₀)alkyl; and 2 to 20-member heteroalkyl as defined above. In another embodiment of the first aspect of the invention, the nanoobject comprises from 1 to 99%, from 20 to 80% or from 30 to 70% by weight of molecules of formula (I) with respect to the total weight of the nanoobject, when: A is —OH, B is —OH or H, and X is selected from (C₁-C₁₀)alkyl and a 2 to 10-member heteroalkyl having from 2 to 10 members, being at least one of the members selected from O, S, and NH, and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH, B is —OH or H, and X is selected from (C₁-C₆)alkyl and a 2 to 6-member heteroalkyl having from 2 to 6 members, being at least one of the members selected from O, S, and NH, and the remaining members being CH₂ members. In another embodiment the nanoobject comprises from 1 to 99%, from 20 to 80% or from 30 to 70% by weight of molecules of formula (I) with respect to the total weight of the nanoobject, when: A is —OH, B is —OH or H, and X is selected from (C₁-C₁₀)alkyl or a 2 to 10-member heteroalkyl having from 2 to 10 members, being at least one of the members selected from O, and NH, and the remaining members are CH₂ members. In another embodiment the nanoobject comprises from 1 to 99%, from 20 to 80% or from 30 to 70% by weight of molecules of formula (I) with respect to the total weight of the nanoobject, when: A is —OH, B is —OH or H, and X is selected from (C₁-C₆)alkyl or a 2 to 6-member heteroalkyl having from 2 to 6 members, being at least one of the members selected from 0, and NH, and the remaining members are CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH, B is —OH or H, and X is a biradical selected from (C₁-C₁₀)alkyl, and a 2 to 10-member heteroalkyl having from 2 to 10 members, being one or two of the members independently selected from O, and NH, and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH, B is —OH or H, and X is a biradical selected from (C₁-C₆)alkyl, and a 2 to 6-member heteroalkyl having from 2 to 6 members, being one or two of the members independently selected from O, and NH, and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH, B is —OH or H, and X is a biradical selected from (C₁-C₁₀)alkyl, and a 2 to 10-member heteroalkyl having from 2 to 10 members, being one or two of them O member(s), and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH, B is —OH or H, and X is a biradical selected from (C₁-C₆)alkyl, and a 2 to 6-member heteroalkyl having from 2 to 6 members, being one or two of them O member(s), and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH, B is —OH or H; and X is a biradical selected from (C₁-C₁₀)alkyl, and a 2 to 10-member heteroalkyl having from 2 to 10 members, being one of them a NH member, and the remaining members being CH₂ members. In another embodiment of the first aspect of the invention, the nanoobject comprises from 1 to 99%, from 20 to 80% or from 30 to 70% by weight of molecules of formula (I) with respect to the total weight of the nanoobject, when: A is —OH, B is —OH or H; and X is a biradical selected from (C₁-C₆)alkyl, and a 2 to 6-member heteroalkyl having from 2 to 6 members, being one of them a NH member, and the remaining members being CH₂ members.

In another embodiment of the first aspect of the invention, the nanoobject comprises from 1 to 99%, from 30 to 99% or from 90 to 99% by weight of molecules of formula (I) with respect to the total weight of the nanoobject when: A is —OH, B is —OH or H, and X is a homopolymer or copolymer as defined in the first aspect of the invention. In another embodiment, the nanoobject comprises from 1 to 99%, from 30 to 99% or from 90 to 99% by weight of molecules of formula (I) with respect to the total weight of the nanoobject, when: A is —OH, B is —OH or H, and X is copolymer or homopolymer comprising a polyether chain. In another embodiment of the first aspect of the invention, the nanoobject comprises from 1 to 99%, from 30 to 99% or from 90 to 99% by weight of molecules of formula (I) with respect to the total weight of the nanoobject when: A is —OH, B is —OH or H, and X is a homopolymer or copolymer comprising a polyethylene oxide chain.

In another embodiment of the first aspect of the invention, the nanoobject is a MoS₂ nanoobject comprising from 1 to 99%, from 15 to 99%, from 20 to 80% or from 30 to 70% by weight of molecules of formula (I) with respect to the total weight of the nanoobject, when: A is —OH, B is (C₁-C₄)alkyl, NH₂, —OH or H, and X is a biradical selected from the group consisting of: (C₁-C₂₀)alkyl; and 2 to 20-member heteroalkyl as defined above. In another embodiment of the first aspect of the invention, the nanoobject is a MoS₂ nanoobject comprising from 1 to 99%, from 15 to 99%, from 20 to 80% or from 30 to 70% by weight of molecules of formula (I) with respect to the total weight of the nanoobject, when: A is —OH, B is (C₁-C₄), NH₂, —OH or H; and X is selected from (C₁-C₁₀)alkyl and a 2 to 10-member heteroalkyl having from 2 to 10 members, being at least one of the members selected from O, S, and NH, and the remaining members are CH₂ members. In another embodiment of the first aspect of the invention, the nanoobject is a MoS₂ nanoobject comprising from 1 to 99%, from 15 to 99%, from 20 to 80% or from 30 to 70% by weight of molecules of formula (I) with respect to the total weight of the nanoobject, when: A is —OH, B is (C₁-C₄), NH₂, —OH or H; and X is selected from (C₁-C₆)alkyl and a 2 to 6-member heteroalkyl having from 2 to 6 members, being at least one of the members selected from O, S, and NH, and the remaining members are CH₂ members. In another embodiment the nanoobject is a MoS₂ nanoobject comprising from 1 to 99%, from 15 to 99%, from 20 to 80% or from 30 to 70% by weight of molecules of formula (I) with respect to the total weight of the nanoobject, when: A is —OH, B is (C₁-C₄), NH₂, —OH or H, and X is selected from (C₁-C₁₀)alkyl or a 2 to 10-member heteroalkyl having from 2 to 10 members, being at least one of the members selected from O, and NH, and the remaining members being CH₂ members. In another embodiment the nanoobject is a MoS₂ nanoobject comprising from 1 to 99%, from 15 to 99%, from 20 to 80% or from 30 to 70% by weight of molecules of formula (I) with respect to the total weight of the nanoobject, when: A is —OH, B is (C₁-C₄), NH₂, —OH or H; and X is selected from (C₁-C₆)alkyl or a 2 to 6-member heteroalkyl having from 2 to 6 members, being at least one of the members selected from O, and NH, and the remaining members being CH₂ members. In another embodiment of the first aspect of the invention, the nanoobject is a MoS₂ nanoobject comprising from 1 to 99%, from 15 to 99%, from 20 to 80% or from 30 to 70% by weight of molecules of formula (I) with respect to the total weight of the nanoobject, when: A is —OH, B is (C₁-C₄), NH₂, —OH or H, and X is a biradical selected from (C₁-C₁₀)alkyl, and a 2 to 10-member heteroalkyl having from 2 to 10 members, being one or two of the members independently selected from O, and NH, and the remaining members being CH₂ members. In another embodiment of the first aspect of the invention, the nanoobject is a MoS₂ nanoobject comprising from 1 to 99%, from 15 to 99%, from 20 to 80% or from 30 to 70% by weight of molecules of formula (I) with respect to the total weight of the nanoobject, when: A is —OH, B is (C₁-C₄), NH₂, —OH or H, and X is a biradical selected from (C₁-C₆)alkyl, and a 2 to 6-member heteroalkyl having from 2 to 6 members, being one or two of the members independently selected from O, and NH, and the remaining members being CH₂ members. In another embodiment of the first aspect of the invention, the nanoobject is a MoS₂ nanoobject comprising from 1 to 99%, from 15 to 99%, from 20 to 80% or from 30 to 70% by weight of molecules of formula (I) with respect to the total weight of the nanoobject, when: A is —OH, B is (C₁-C₄), NH₂, —OH or H, and X is a biradical X selected from (C₁-C₁₀)alkyl, and a 2 to 10-member heteroalkyl having from 2 to 10 members, being one or two of them O member(s), and the remaining members being CH₂ members. In another embodiment of the first aspect of the invention, the nanoobject is a MoS₂ nanoobject comprising from 1 to 99%, from 15 to 99%, from 20 to 80% or from 30 to 70% by weight of molecules of formula (I) with respect to the total weight of the nanoobject, when: A is —OH, B is (C₁-C₄), NH₂, —OH or H, and X is a biradical X selected from (C₁-C₆)alkyl, and a 2 to 6-member heteroalkyl having from 2 to 6 members, being one or two of them a O member, and the remaining members being CH₂ members. In another embodiment of the first aspect of the invention, the nanoobject is a MoS₂ nanoobject comprising from 1 to 99%, from 15 to 99%, from 20 to 80% or from 30 to 70% by weight of molecules of formula (I) with respect to the total weight of the nanoobject, when: A is —OH, B is (C₁-C₄), NH₂, —OH or H, and X is a biradical selected from (C₁-C₁₀)alkyl, and a 2 to 10-member heteroalkyl having from 2 to 10 members, being one of them a NH member, and the remaining members being CH₂ members. In another embodiment of the first aspect of the invention, the nanoobject is a MoS₂ nanoobject comprising from 1 to 99%, from 15 to 99%, from 20 to 80% or from 30 to 70% by weight of molecules of formula (I) with respect to the total weight of the nanoobject, when: A is —OH, B is (C₁-C₄), NH₂, —OH or H; and X is a biradical selected from (C₁-C₆)alkyl, and a 2 to 6-member heteroalkyl having from 2 to 6 members, being one of them a NH member, and the remaining members being CH₂ members.

In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH or —SH, B is —OH or H, and X is a biradical selected from the group consisting of: (C₁-C₂₀)alkyl; and 2 to 20-member heteroalkyl as defined above. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH or —SH, B is —OH or H; and X is selected from (C₁-C₁₀)alkyl and a 2 to 10-member heteroalkyl having from 2 to 10 members, being at least one of the members selected from O, S, and NH, and the remaining members are CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH or —SH, B is —OH or H; and X is selected from (C₁-C₆)alkyl and a 2 to 6-member heteroalkyl having from 2 to 6 members, being at least one of the members selected from O, S, and NH, and the remaining members are CH₂ members. In another embodiment the nanoobject is a MoS₂ nanoobject comprising from 1 to 99%, from 20 to 80% or from 30 to 70% by weight of molecules of formula (I) with respect to the total weight of the nanoobject, when: A is —OH or —SH, B is —OH or H, and X is selected from (C₁-C₁₀)alkyl or a 2 to 10-member heteroalkyl having from 2 to 10 members, being at least one of the members selected from O, and NH, and the remaining members being CH₂ members. In another embodiment the nanoobject is a MoS₂ nanoobject comprising from 1 to 99%, from 20 to 80% or from 30 to 70% by weight of molecules of formula (I) with respect to the total weight of the nanoobject, when: A is —OH or —SH, B is —OH or H; and X is selected from (C₁-C₆)alkyl or a 2 to 6-member heteroalkyl having from 2 to 6 members, being at least one of the members selected from O, and NH, and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH or —SH, B is —OH or H, and X is a biradical selected from (C₁-C₁₀)alkyl, and a 2 to 10-member heteroalkyl having from 2 to 10 members, being one or two of the members independently selected from O, and NH, and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH or —SH, B is —OH or H, and X is a biradical selected from (C₁-C₆)alkyl, and a 2 to 6-member heteroalkyl having from 2 to 6 members, being one or two of the members independently selected from O, and NH, and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH or —SH, B is —OH or H, and X is a biradical X selected from (C₁-C₁₀)alkyl, and a 2 to 10-member heteroalkyl having from 2 to 10 members, being one or two of them O member(s), and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH or —SH, B is —OH or H, and X is a biradical X selected from (C₁-C₆)alkyl, and a 2 to 6-member heteroalkyl having from 2 to 6 members, being one or two of them a O member, and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH or —SH, B is —OH or H, and X is a biradical selected from (C₁-C₁₀)alkyl, and a 2 to 10-member heteroalkyl having from 2 to 10 members, being one of them a NH member, and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH or —SH, B is —OH or H; and X is a biradical selected from (C₁-C₆)alkyl, and a 2 to 6-member heteroalkyl having from 2 to 6 members, being one of them a NH member, and the remaining members being CH₂ members.

In another embodiment of the first aspect of the invention, the nanoobject is a MoS₂ nanoobject comprising from 1 to 99%, from 30 to 99% or from 90 to 99% by weight of molecules of formula (I) with respect to the total weight of the nanoobject when: A is —OH or —SH, B is —OH or H, and X is a homopolymer or copolymer as defined in the first aspect of the invention. In another embodiment, the nanoobject is a MoS₂ nanoobject comprising from 1 to 99%, from 30 to 99% or from 90 to 99% by weight of molecules of formula (I) with respect to the total weight of the nanoobject, when: A is —OH or —SH, B is —OH or H, and X is copolymer or homopolymer comprising a polyether chain. In another embodiment of the first aspect of the invention, the nanoobject is a MoS₂ nanoobject comprising from 1 to 99%, from 30 to 99% or from 90 to 99% by weight of molecules of formula (I) with respect to the total weight of the nanoobject when: A is —OH or —SH, B is —OH or H, and X is a homopolymer or copolymer comprising a polyethylene oxide chain.

In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH, B is —OH or H, and X is a biradical selected from the group consisting of: (C₁-C₂₀)alkyl; and 2 to 20-member heteroalkyl as defined above. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH, B is —OH or H, and X is selected from (C₁-C₁₀)alkyl and a 2 to 10-member heteroalkyl having from 2 to 10 members, being at least one of the members selected from O, S, and NH, and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH, B is —OH or H, and X is selected from (C₁-C₆)alkyl and a 2 to 6-member heteroalkyl having from 2 to 6 members, being at least one of the members selected from O, S, and NH, and the remaining members being CH₂ members. In another embodiment the nanoobject is a MoS₂ nanoobject comprising from 1 to 99%, from 20 to 80% or from 30 to 70% by weight of molecules of formula (I) with respect to the total weight of the nanoobject, when: A is —OH, B is —OH or H, and X is selected from (C₁-C₁₀)alkyl or a 2 to 10-member heteroalkyl having from 2 to 10 members, being at least one of the members selected from O, and NH, and the remaining members being CH₂ members. In another embodiment the nanoobject is a MoS₂ nanoobject comprising from 1 to 99%, from 20 to 80% or from 30 to 70% by weight of molecules of formula (I) with respect to the total weight of the nanoobject, when: A is —OH, B is —OH or H; and X is selected from (C₁-C₆)alkyl or a 2 to 6-member heteroalkyl having from 2 to 6 members, being at least one of the members selected from O, and NH, and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH, B is —OH or H, and X is a biradical selected from (C₁-C₆)alkyl, and a 2 to 10-member heteroalkyl having from 2 to 10 members, being one or two of the members independently selected from O, and NH, and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH, B is —OH or H, and X is a biradical selected from (C₁-C₆)alkyl, and a 2 to 6-member heteroalkyl having from 2 to 6 members, being one or two of the members independently selected from O, and NH, and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH, B is —OH or H and X is a biradical selected from (C₁-C₁₀)alkyl, and a 2 to 10-member heteroalkyl having from 2 to 10 members, being one or two of them O member(s), and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH, B is —OH or H, and X is a biradical selected from (C₁-C₆)alkyl, and a 2 to 6-member heteroalkyl having from 2 to 6 members, being one or two of them O member(s), and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH, B is —OH or H, and X is a biradical selected from (C₁-C₁₀)alkyl, and a 2 to 10-member heteroalkyl having from 2 to 10 members, being one of them a NH member, and the remaining members being CH₂ members. In another embodiment of the first aspect of the invention, the nanoobject is a MoS₂ nanoobject comprising from 1 to 99%, from 20 to 80% or from 30 to 70% by weight of molecules of formula (I) with respect to the total weight of the nanoobject, when: A is —OH, B is —OH or H; and X is a biradical selected from (C₁-C₆)alkyl, and a 2 to 6-member heteroalkyl having from 2 to 6 members, being one of them a NH member, and the remaining members being CH₂ members.

In another embodiment of the first aspect of the invention, the nanoobject is a MoS₂ nanoobject comprising from 1 to 99%, from 30 to 99% or from 90 to 99% by weight of molecules of formula (I) with respect to the total weight of the nanoobject when: A is —OH, B is —OH or H, and X is a homopolymer or copolymer as defined in the first aspect of the invention. In another embodiment, the nanoobject is a MoS₂ nanoobject comprising from 1 to 99%, from 30 to 99% or from 90 to 99% by weight of molecules of formula (I) with respect to the total weight of the nanoobject, when: A is —OH, B is —OH or H, and X is copolymer or homopolymer comprising a polyether chain. In another embodiment of the first aspect of the invention, the nanoobject is a MoS₂ nanoobject comprising from 1 to 99%, from 30 to 99% or from 90 to 99% by weight of molecules of formula (I) with respect to the total weight of the nanoobject when: A is —OH, B is —OH or H, and X is a homopolymer or copolymer comprising a polyethylene oxide chain.

In another embodiment of the first aspect of the invention the object size is comprised from 0.1 to 500 nm. In another embodiment, when the nanoobject is spherical, the object size is comprised from 10 to 500 nm, from 20 to 250 nm or from 30 to 100 nm. In another embodiment, when the nanoobject has box-shape, the object size is comprised from 0.1 to 50 nm, from 0.2 to 30 nm or from 0.3 to 15 nm. In another embodiment, when the nanoobject has rod-shape, the object size is comprised from 1 to 100 nm, from 5 to 50 nm or from 10 to 30 nm.

In another embodiment of the first aspect of the invention, the nanoobject comprises a single type of molecule of formula (I). This means, for instance, that the surface of the nanoobject is functionalized with uniquely propylene glycol molecules, or alternatively by ethylene glycol molecules, or alternatively by diethylene glycol molecules, or alternatively by polyethylene glycol molecules, or alternatively by polyethylene glycol monomethyl ether, or alternatively by diethanolamine molecules, or alternatively by 1,6-hexanediol molecules, or alternatively by 6-amino-1-hexanol molecules.

In another embodiment of the first aspect of the invention, the surface the nanoobject comprises different molecules of formula (I). This means that the surface of the nanoobject can be functionalized with a mixture of two or more different molecules of formula (I), such as propylene glycol molecules plus diethylene glycol molecules, or polyethylene glycol molecules plus 1,6-hexanediol molecules.

In a second aspect the present invention provides a process for preparing the nanoobject as defined in the first aspect of the invention.

The term “Molybdenum source” encompasses an alkali or alkali earth molybdate such as sodium molybdate, potassium molybdate, calcium molybdate, cesium molybdate, zinc molybdate, lithium molybdate and the like; metallic molybdates such as aluminium molybdate, iron (II) molybdate, silver molybdate, manganese (II) molybdate and the like; ammonium molybdate; ammonium orthomolybdate; ammonium phosphomolybdate; ammonium tetrathiomolybdate; molybdic acid, elemental molybdenum; molybdenum disilicide; molybdenum trisulfide; molybdenum halides such as molybdenum hexafluoride and molybdenum tetrachloride and the like; or various oxides of molybdenum such as molybdenum dioxide, trioxide, and the like. In one embodiment, the molybdenum salt is an alkali or alkali earth molybdate, such as sodium molybdate, potassium molybdate, calcium molybdate cesium molybdate, zinc molybdate, lithium molybdate or the like; ammonium molybdate; ammonium orthomolybdate; ammonium phosphomolydate; ammonium tetrathiomolybdate; or one of the various oxides of molybdenum such as molybdenum dioxide, trioxide, and the like. In one embodiment, the molybdenum source is a molybdenum salt. In another embodiment the molybdenum salt is selected from sodium molybdate, potassium molybdate, ammonium molybdate, and ammonium tetrathiomolybdate.

The term “Tungsten source” encompasses alkali and alkali earth tungstate such as sodium tungstate, potassium tungstate, calcium tungstate, cesium tungstate, zinc tungstate, lithium tungstate and the like; metallic tungstate such as aluminium tungstate, iron (II) tungstate, silver tungstate, manganese (II) tungstate and the like; ammonium tungstate; ammonium orthotungstate; ammonium phosphotungstate; ammonium tetrathiotungstate; tustenic acid; elemental tungsten, tungsten disilicide; tungsten trisulfide; tungsten halides such as tungsten hexafluoride and tungsten tetrachloride and the like; or one of the various oxides of tungsten such as tungsten dioxide, trioxide, and the like. In another embodiment the tungstate salt can be an alkali or alkali earth tungstate such as sodium tungstate, potassium tungstate, calcium tungstate cesium tungstate, zinc tungstate, lithium tungstate and the like; or alternatively it can be ammonium tungstate, ammonium tungstate, ammonium phosphotungstate, ammonium tetrathiotungstate, or one of the various oxides of tungsten such as tungsten dioxide, trioxide, and the like. In one embodiment the tungsten source is a tungsten salt. In another embodiment, the tungstate salt can be sodium tungstate, potassium tungstate, ammonium tungstate, ammonium tetrathiotungstate or the like.

The term “Sulfur source” encompasses alkali and alkali earth sulfate such as sodium sulfate, potassium sulfate, zinc sulfate, calcium sulfate and the like; metallic sulfates such as aluminium sulfate, copper (I, II) sulfate, ferrous sulfate (II), cobalt (II) sulfate and the like; ammonium sulfate, ammonium thiosulfate, sodium thiosulfate, thioamides, thioacetamides, and thioureas; elemental sulfur, ammonium sulfide, sodium sulfide, potassium sulfide, carbon disulfide and the like. In one embodiment, the sulfur source is selected from sodium sulfate, potassium sulfate, ammonium sulfate, ammonium thiosulfate, sodium thiosulfate, thioureas, ammonium sulfide, sodium sulfide and potassium sulfide. In another embodiment, the sulfur source is ammonium thiosulfate, sodium thiosulfate or a thiourea.

In the present invention the term “selenium source” encompasses alkali and alkali earth selenate such as sodium selenate, potassium selenate, zinc selenate, calcium selenate and the like; metallic selenates such as aluminium selenate, copper (I, II) selenate, ferrous selenate (II), cobalt (II) selenate and the like; ammonium selenate, ammonium selenosulfate, sodium selenosulfate, selenoamides, selenoacetamides and selenoureas; elemental selenium, ammonium selenide, sodium selenide, potassium selenide, carbon diselenide and the like. In one embodiment, the selenium source is selected from sodium selenate, potassium selenate, ammonium selenate, ammonium selenosulfate, sodium selenosulfate, selenoureas, ammonium selenide, sodium selenide and potassium selenide. In another embodiment, the selenium source is selected from ammonium selenosulfate, sodium selenosulfate and selenoureas.

In the present invention the term “tellurium sources” encompasses alkali and alkali earth tellurate such as sodium tellurate, potassium tellurate, zinc tellurate, calcium tellurate and the like; metallic tellurate such as aluminium tellurate, copper (I, II) tellurate, ferrous tellurate (II), cobalt (II) tellurate and the like; ammonium tellurate, ammonium tellurosulfate, sodium tellurosulfate, telluroamides, telluroacetamides and telluroureas; elemental tellurium, ammonium telluride, sodium telluride, potassium telluride, carbon ditelluride and the like. In one embodiment, the tellurium source is selected from sodium tellurate, potassium tellurate, ammonium tellurate, ammonium tellurosulfate, sodium tellurosulfate, telluroureas, ammonium telluride, sodium telluride and potassium telluride. In another embodiment, the tellurium source is selected from ammonium tellurosulfate, sodium tellurosulfate, and telluroureas.

All embodiments concerning the molecules of formula (I) as well as concerning the nanoobjects of the first aspect of the invention, also applies for the process of the second aspect wherein it is provided a method for preparing nanoobjects using such molecules.

In one embodiment of the second aspect, the process comprises the step of reacting a Molybdate or Tungstate salt with an urea compound selected from the group consisting of: NR₈R₉—C(═S)—NR₁₀R₁₁, NR₈R₉—C(═Se)—NR₁₀R₁₁, and NR₈R₉—C(═Te)—NR₁₀R₁₁, where R₈ to R₁₁ are selected from of H, (C₁-C₂₀)alkyl, (C₅-C₁₂)aryl(C₁-C₂₀)alkyl and (C₅-C₁₂)aryl. The invention also provides a product obtainable following the process of this embodiment.

In one embodiment of the second aspect of the invention, the salt is a Molybdate salt, and the urea compound and the molecule of formula (I) are as defined above.

In another embodiment of the second aspect of the invention, the process comprises reacting a Molybdate salt with NR₈R₉—C(═S)—NR₁₀R₁₁, being R₈-R₁₁ as defined above, and the molecule of formula (I) being as defined in any of the above embodiments. In another embodiment of the second aspect of the invention, the process comprises reacting a Molybdate salt with NH₂—C(═S)—NH₂, and the molecules of formula (I) is as defined in any one of the above embodiments. The present invention also provides a product obtainable following the process of this embodiment.

In another embodiment of the second aspect of the invention, the process comprises reacting a Molybdate salt with NH₂—C(═S)—NH₂, and the molecules of formula (I) is one wherein A is —OH, and B and X are as defined above. The present invention also provides a product obtainable following the process of this embodiment.

In another embodiment of the second aspect of the invention, the process comprises reacting a Molybdate salt with NH₂—C(═S)—NH₂, and the molecules of formula (I) is one wherein A is —OH, B is (C₁-C₄) alkyl, NH₂, —OH or H, and X is selected from: (C₁-C₁₀)alkyl; a 2- to 10-member heteroalkyl having from 2 to 10 members, being at least one of the members selected from O, S, and NH, and the remaining members being CH₂ members; and a homopolymer or copolymer having a polymeric chain selected from the group consisting of: alkyd resin, epoxy resin, phenolic resin, polyvinyl halides, polyacetal, polyacrylics, polyalkylenes, polyalkenylenes, polyalkynylenes, polyamides, polyamines, polyanhydrides, polycarbonates, polyester-polyurethanes, polyesters, polyetheretherketones, polyether-polyurethanes, polyethers, polyimidazoles, polyimides, polyisocyanurates, polyketones, polyolefines, polyoxyalkylenes, polyoxyphenylenes, polypyrroles, polysiloxanes, polysulfides, polysulfonamides, polysulfones, polythiazoles, polythiomethylenes, polythiophenylenes, polyureas, polyurethanes, polyvinyl acetals, polyvinyl butyrals, polyvinyl formals, polyvinyl alkanoates, vinyl polymers, and natural polymers; provided that when B is —NH₂, then X is selected from: (C₁-C₁₀)alkyl; and a 2- to 10-member heteroalkyl having from 2 to 10 members, being at least one of the members selected from O, S, and NH, and the remaining members being CH₂ members. The present invention also provides a product obtainable following the process of this embodiment.

In another embodiment of the second aspect of the invention, the process comprises reacting a Molybdate salt with NH₂—C(═S)—NH₂, and the molecules of formula (I) is one wherein A is —OH, B is —OH or H, and X is selected from: (C₁-C₁₀)alkyl; a 2- to 10-member heteroalkyl having from 2 to 10 members, being at least one of the members selected from O, S, and NH, and the remaining members being CH₂ members; and a homopolymer or copolymer having a polymeric chain selected from the group consisting of: alkyd resin, epoxy resin, phenolic resin, polyvinyl halides, polyacetal, polyacrylics, polyalkylenes, polyalkenylenes, polyalkynylenes, polyamides, polyamines, polyanhydrides, polycarbonates, polyester-polyurethanes, polyesters, polyetheretherketones, polyether-polyurethanes, polyethers, polyimidazoles, polyimides, polyisocyanurates, polyketones, polyolefines, polyoxyalkylenes, polyoxyphenylenes, polypyrroles, polysiloxanes, polysulfides, polysulfonamides, polysulfones, polythiazoles, polythiomethylenes, polythiophenylenes, polyureas, polyurethanes, polyvinyl acetals, polyvinyl butyrals, polyvinyl formals polyvinyl alkanoates, vinyl polymers, and natural polymers. The present invention also provides a product obtainable following the process of this embodiment.

In another embodiment of the second aspect of the invention, the process comprises reacting a Molybdate salt with NH₂—C(═S)—NH₂, and the molecules of formula (I) is one wherein A is —OH, B is (C₁-C₄) alkyl, NH₂, —OH or H, and X is selected from: (C₁-C₆)alkyl; a 2- to 6-member heteroalkyl having from 2 to 6 members, being at least one of the members selected from O, S, and NH, and the remaining members being CH₂ members; and a homopolymer or copolymer having a polymeric chain selected from the group consisting of: alkyd resin, epoxy resin, phenolic resin, polyvinyl halides, polyacetal, polyacrylics, polyalkylenes, polyalkenylenes, polyalkynylenes, polyamides, polyamines, polyanhydrides, polycarbonates, polyester-polyurethanes, polyesters, polyetheretherketones, polyether-polyurethanes, polyethers, polyimidazoles, polyimides, polyisocyanurates, polyketones, polyolefines, polyoxyalkylenes, polyoxyphenylenes, polypyrroles, polysiloxanes, polysulfides, polysulfonamides, polysulfones, polythiazoles, polythiomethylenes, polythiophenylenes, polyureas, polyurethanes, polyvinyl acetals, polyvinyl butyrals, polyvinyl formals, polyvinyl alkanoates, vinyl polymers, and natural polymers; provided that when B is —NH₂, then X is selected from (C₁-C₆)alkyl; and a 2- to 6-member heteroalkyl having from 2 to 6 members, being at least one of the members selected from O, S, and NH, and the remaining members being CH₂ members. The present invention also provides a product obtainable following the process of this embodiment.

In another embodiment of the second aspect of the invention, the process comprises reacting a Molybdate salt with NH₂—C(═S)—NH₂, and the molecules of formula (I) is one wherein A is —OH, B is —OH or H, and X is selected from: (C₁-C₆)alkyl; a 2- to 6-member heteroalkyl having from 2 to 6 members, being at least one of the members selected from O, S, and NH, and the remaining members being CH₂ members; and a homopolymer or copolymer having a polymeric chain selected from the group consisting of: alkyd resin, epoxy resin, phenolic resin, polyvinyl halides, polyacetal, polyacrylics, polyalkylenes, polyalkenylenes, polyalkynylenes, polyamides, polyamines, polyanhydrides, polycarbonates, polyester-polyurethanes, polyesters, polyetheretherketones, polyether-polyurethanes, polyethers, polyimidazoles, polyimides, polyisocyanurates, polyketones, polyolefines, polyoxyalkylenes, polyoxyphenylenes, polypyrroles, polysiloxanes, polysulfides, polysulfonamides, polysulfones, polythiazoles, polythiomethylenes, polythiophenylenes, polyureas, polyurethanes, polyvinyl acetals, polyvinyl butyrals, polyvinyl formals polyvinyl alkanoates, vinyl polymers, and natural polymers. The present invention also provides a product obtainable following the process of this embodiment.

In another embodiment of the second aspect of the invention the process comprises reacting a Molybdate salt with NH₂—C(═S)—NH₂, and molecules of formula (I) wherein A is —OH, B is (C₁-C₄) alkyl, NH₂, —OH or H, and X is selected from: (C₁-C₁₀)alkyl; a 2- to 10-member heteroalkyl having from 2 to 10 members, being at least one of the members selected from O, S, and NH, and the remaining members being CH₂ members; and a homopolymer or copolymer comprising a polymeric chain selected from epoxy resin, phenolic resin, polyvinyl halides, polyacetal, polyacrylics, polyamides, polyamines, polycarbonates, polyester-polyurethanes, polyesters, polyether-polyurethanes, polyethers, polyimides, polyketones, polyolefines, polyoxyalkylenes, polyoxyphenylenes, polysiloxanes, polysulfides, polysulfones, polythiomethylenes, polyureas, polyurethanes, polyvinyl acetals, polyvinyl alkanoates, and natural polymers; provided that when B is —NH₂, then X is selected from: (C₁-C₁₀)alkyl; and a 2- to 10-member heteroalkyl having from 2 to 10 members, being at least one of the members selected from O, S, and NH, and the remaining members being CH₂ members. The present invention also provides a product obtainable following the process of this embodiment.

In another embodiment of the second aspect of the invention the process comprises reacting a Molybdate salt with NH₂—C(═S)—NH₂, and molecules of formula (I) wherein A is —OH, B is —OH or H, and X is selected from: (C₁-C₁₀)alkyl; a 2- to 10-member heteroalkyl having from 2 to 10 members, being at least one of the members selected from O, S, and NH, and the remaining members being CH₂ members; and a homopolymer or copolymer comprising a polymeric chain selected from epoxy resin, phenolic resin, polyvinyl halides, polyacetal, polyacrylics, polyamides, polyamines, polycarbonates, polyester-polyurethanes, polyesters, polyether-polyurethanes, polyethers, polyimides, polyketones, polyolefines, polyoxyalkylenes, polyoxyphenylenes, polysiloxanes, polysulfides, polysulfones, polythiomethylenes, polyureas, polyurethanes, polyvinyl acetals, polyvinyl alkanoates, and natural polymers. The present invention also provides a product obtainable following the process of this embodiment.

In another embodiment of the second aspect of the invention the process comprises reacting a Molybdate salt with NH₂—C(═S)—NH₂, and molecules of formula (I) wherein A is —OH, B is (C₁-C₄) alkyl, NH₂, —OH or H, and X is selected from: (C₁-C₆)alkyl; a 2- to 6-member heteroalkyl having from 2 to 6 members, being at least one of the members selected from O, S, and NH, and the remaining members being CH₂ members; and a homopolymer or copolymer comprising a polymeric chain selected from epoxy resin, phenolic resin, polyvinyl halides, polyacetal, polyacrylics, polyamides, polyamines, polycarbonates, polyester-polyurethanes, polyesters, polyether-polyurethanes, polyethers, polyimides, polyketones, polyolefines, polyoxyalkylenes, polyoxyphenylenes, polysiloxanes, polysulfides, polysulfones, polythiomethylenes, polyureas, polyurethanes, polyvinyl acetals, polyvinyl alkanoates, and natural polymers; provided that when B is —NH₂, then X is selected from: (C₁-C₆)alkyl; and a 2- to 6-member heteroalkyl having from 2 to 6 members, being at least one of the members selected from O, S, and NH, and the remaining members being CH₂ members. The present invention also provides a product obtainable following the process of this embodiment.

In another embodiment of the second aspect of the invention the process comprises reacting a Molybdate salt with NH₂—C(═S)—NH₂, and molecules of formula (I) wherein A is —OH, B is —OH or H, and X is selected from: (C₁-C₆)alkyl; a 2- to 6-member heteroalkyl having from 2 to 6 members, being at least one of the members selected from O, S, and NH, and the remaining members being CH₂ members; and a homopolymer or copolymer comprising a polymeric chain selected from epoxy resin, phenolic resin, polyvinyl halides, polyacetal, polyacrylics, polyamides, polyamines, polycarbonates, polyester-polyurethanes, polyesters, polyether-polyurethanes, polyethers, polyimides, polyketones, polyolefines, polyoxyalkylenes, polyoxyphenylenes, polysiloxanes, polysulfides, polysulfones, polythiomethylenes, polyureas, polyurethanes, polyvinyl acetals, and polyvinyl alkanoates, and natural polymers. The present invention also provides a product obtainable following the process of this embodiment.

In another embodiment of the second aspect of the invention, the process comprises reacting a Molybdate salt with NH₂—C(═S)—NH₂, and molecules of formula (I) wherein A is —OH, B is (C₁-C₄) alkyl, NH₂, —OH or H, and X is selected from: (C₁-C₁₀)alkyl; a 2- to 10-member heteroalkyl having from 2 to 10 members, being at least one of the members selected from O, S, and NH, and the remaining members being CH₂ members; and a homopolymer o copolymer comprising a polyether chain; provided that when B is —NH₂, then X is selected from: (C₁-C₁₀)alkyl; and a 2- to 10-member heteroalkyl having from 2 to 10 members, being at least one of the members selected from O, S, and NH, and the remaining members being CH₂ members. The present invention also provides a product obtainable following the process of this embodiment.

In another embodiment of the second aspect of the invention, the process comprises reacting a Molybdate salt with NH₂—C(═S)—NH₂, and molecules of formula (I) wherein A is —OH, B is —OH or H, and X is selected from: (C₁-C₁₀)alkyl; a 2- to 10-member heteroalkyl having from 2 to 10 members, being at least one of the members selected from O, S, and NH, and the remaining members being CH₂ members; and a homopolymer o copolymer comprising a polyether chain. The present invention also provides a product obtainable following the process of this embodiment.

In another embodiment of the second aspect of the invention, the process comprises reacting a Molybdate salt with NH₂—C(═S)—NH₂, and molecules of formula (I) wherein A is —OH, B is (C₁-C₄) alkyl, NH₂, —OH or H, and X is selected from: (C₁-C₆)alkyl; a 2- to 6-member heteroalkyl having from 2 to 6 members, being at least one of the members selected from O, S, and NH, and the remaining members being CH₂ members; and a homopolymer o copolymer comprising a polyether chain; provided that provided that when B is —NH₂, then X is selected from: (C₁-C₆)alkyl; and a 2- to 6-member heteroalkyl having from 2 to 6 members, being at least one of the members selected from O, S, and NH, and the remaining members being CH₂ members. The present invention also provides a product obtainable following the process of this embodiment.

In another embodiment of the second aspect of the invention, the process comprises reacting a Molybdate salt with NH₂—C(═S)—NH₂, and molecules of formula (I) wherein A is —OH, B is —OH or H, and X is selected from: (C₁-C₆)alkyl; a 2- to 6-member heteroalkyl having from 2 to 6 members, being at least one of the members selected from O, S, and NH, and the remaining members being CH₂ members; and a homopolymer o copolymer comprising a polyether chain. The present invention also provides a product obtainable following the process of this embodiment.

In another embodiment of the second aspect of the invention, the process comprises reacting a Molybdate salt with NH₂—C(═S)—NH₂, and molecules of formula (I) wherein A is —OH, B is (C₁-C₄) alkyl, NH₂, —OH or H, and X is selected from: (C₁-C₁₀)alkyl; a 2- to 10-member heteroalkyl having from 2 to 10 members, being at least one of the members selected from O, S, and NH, and the remaining members being CH₂ members; and a homopolymer or copolymer comprising a polyethylene oxide chain; provided that when B is —NH₂, then X is selected from: (C₁-C₁₀)alkyl; and a 2- to 10-member heteroalkyl having from 2 to 10 members, being at least one of the members selected from O, S, and NH, and the remaining members being CH₂ members. The present invention also provides a product obtainable following the process of this embodiment. In another embodiment of the second aspect of the invention, the process comprises reacting a Molybdate salt with NH₂—C(═S)—NH₂, and molecules of formula (I) wherein A is —OH, B is (C₁-C₄) alkyl, NH₂, —OH or H, and X is selected from: (C₁-C₆)alkyl; a 2- to 6-member heteroalkyl having from 2 to 6 members, being at least one of the members selected from O, S, and NH, and the remaining members being CH₂ members; and a homopolymer or copolymer comprising a polyethylene oxide chain; provided that provided that when B is —NH₂, then X is selected from: (C₁-C₆)alkyl; and a 2- to 6-member heteroalkyl having from 2 to 6 members, being at least one of the members selected from O, S, and NH, and the remaining members being CH₂ members. The present invention also provides a product obtainable following the process of this embodiment.

In another embodiment of the second aspect of the invention, the process comprises reacting a Molybdate salt with NH₂—C(═S)—NH₂, and molecules of formula (I) wherein A is —OH, B is —OH or H, and X is selected from: (C₁-C₁₀)alkyl; a 2- to 10-member heteroalkyl having from 2 to 10 members, being at least one of the members selected from O, S, and NH, and the remaining members being CH₂ members; and a homopolymer or copolymer comprising a polyethylene oxide chain. The present invention also provides a product obtainable following the process of this embodiment. In another embodiment of the second aspect of the invention, the process comprises reacting a Molybdate salt with NH₂—C(═S)—NH₂, and molecules of formula (I) wherein A is —OH, B is —OH or H, and X is selected from: (C₁-C₆)alkyl; a 2- to 6-member heteroalkyl having from 2 to 6 members, being at least one of the members selected from O, S, and NH, and the remaining members being CH₂ members; and a homopolymer or copolymer comprising a polyethylene oxide chain. The present invention also provides a product obtainable following the process of this embodiment.

In another embodiment of the second aspect of the invention, the process comprises reacting a Molybdate salt with NH₂—C(═S)—NH₂, and the molecules of formula (I) are independently selected from the group consisting of: propylene glycol, ethylene glycol, diethylene glycol, polyethylene glycol, polyethylene glycol monomethyl ether, diethanolamine, 1,6-hexanediol, and 6-amino-1-hexanol. The present invention also provides a product obtainable following the process of this embodiment.

In any of the embodiments of the process of the second aspect of the invention, the reaction is performed at a temperature comprised from 100 to 400° C., from 140 to 300° C. or from 160 to 250° C.

As it is well-known by those skilled in the art, the specific geometry of the nanoobject will depend on the cooling rate. It is also well-recognized that the skilled person, using the general knowledge, is able to adjust the cooling parameters to obtain a spherical, a rod-shaped or a box-shaped nanoobject.

In a fourth, the present invention provides the use of the nanoobjects of the invention as additive for reducing the friction coefficient of a material.

In one embodiment, the material is an oil lubricant.

Lubricating oils useful in this invention are derived from natural lubricating oils, synthetic lubricating oils, and mixtures thereof. In general, both the natural and synthetic lubricating oil will each have a Kinematic viscosity ranging from about 1 to about 100 mm²/s (cSt) at 100° C., although typical applications will require the lubricating oil or lubricating oil mixture to have a viscosity ranging from about 2 to about 30 mm²/s (cSt) at 100° C.

Natural lubricating oils include animal oils, vegetable oils (e.g., castor oil and lard oil), petroleum oils, mineral oils, and oils derived from coal or shale. The preferred natural lubricating oil is mineral oil.

Suitable mineral oils include all common mineral oil basestocks. This includes oils that are naphthenic or paraffinic in chemical structure. Oils that are refined by conventional methodology using acid, alkali, and clay or other agents such as aluminum chloride, or they may be extracted oils produced, for example, by solvent extraction with solvents such as phenol, sulfur dioxide, furfural, dichlorodiethyl ether, etc. They may be hydrotreated or hydrofined, dewaxed by chilling or catalytic dewaxing processes, or hydrocracked. The mineral oil may be produced from natural crude sources or be composed of isomerized wax materials or residues of other refining processes. Typically the mineral oils will have Kinematic viscosities of from 2.0 mm²/s (cSt) to 30.0 mm²/s (cSt) at 100° C. The preferred mineral oils have Kinematic viscosities of from 3 to 20 mm²/s (cSt), and most preferred are those mineral oils with viscosities of 5 to 15 mm²/s (cSt) at 100° C.

Synthetic lubricating oils include hydrocarbon oils and halo-substituted hydrocarbon oils such as oligomerized, polymerized, and interpolymerized olefins (such as polybutylenes, polypropylenes, propylene, isobutylene copolymers, chlorinated polylactones, poly(1-hexenes), poly(1-octenes), poly-(1-decenes), etc., and mixtures thereof); alkylbenzenes (e.g., dodecyl-benzenes, tetradecylbenzenes, dinonyl-benzenes, di(2-ethylhexyl)benzene, etc.); polyphenyls (e.g., biphenyls, terphenyls, alkylated polyphenyls, etc.); and alkylated diphenyl ethers, alkylated diphenyl sulfides, as well as their derivatives, analogs, and homologs thereof, and the like. The preferred oils from this class of synthetic oils are oligomers of [alpha]-olefins, particularly oligomers of 1-decene. Synthetic lubricating oils also include alkylene oxide polymers, interpolymers, copolymers, and derivatives thereof where the terminal hydroxyl groups have been modified by esterification, etherification, etc. This class of synthetic oils is exemplified by: polyoxyalkylene polymers prepared by polymerization of ethylene oxide or propylene oxide; the alkyl and aryl ethers of these polyoxyalkylene polymers (e.g., methyl-polyisopropylene glycol ether having an average molecular weight of 1000, diphenyl ether of polypropylene glycol having a molecular weight of 1000 to 1500); and mono- and poly-carboxylic esters thereof (e.g., the acetic acid esters, mixed C₃-C₈ fatty acid esters, and C₁₂ oxo acid diester of tetraethylene glycol). Another suitable class of synthetic lubricating oils comprises the esters of dicarboxylic acids (e.g., phthalic acid, succinic acid, alkyl succinic acids and alkenyl succinic acids, maleic acid, azelaic acid, suberic acid, sebasic acid, fumaric acid, adipic acid, linoleic acid dimer, malonic acid, alkylmalonic acids, alkenyl malonic acids, etc.) with a variety of alcohols (e.g., butyl alcohol, hexyl alcohol, dodecyl alcohol, 2-ethylhexyl alcohol, ethylene glycol, diethylene glycol monoethers, propylene glycol, etc.). Specific examples of these esters include dibutyl adipate, di(2-ethylhexyl) sebacate, di-n-hexyl fumarate, dioctyl sebacate, diisooctyl azelate, diisodecyl azelate, dioctyl phthalate, didecyl phthalate, dieicosyl sebacate, the 2-ethylhexyl diester of linoleic acid dimer, and the complex ester formed by reacting one mole of sebasic acid with two moles of tetraethylene glycol and two moles of 2-ethyl-hexanoic acid, and the like. Esters useful as synthetic lubricating oils also include those made from C₅ to C₁₂ monocarboxylic acids and polyols and polyol ethers such as neopentyl glycol, trimethylolpropane pentaerythritol, dipentaerythritol, tripentaerythritol, and the like. Silicon-based oils (such as the polyalkyl-, polyaryl-, polyalkoxy-, or polyaryloxy-siloxane oils and silicate oils) comprise another useful class of synthetic lubricating oils. These oils include tetraethyl silicate, tetraisopropyl silicate, tetra-(2-ethylhexyl)silicate, tetra-(4-methyl-2-ethylhexyl)silicate, tetra-(p-tert-butylphenyl)silicate, hexa-(4-methyl-2-pentoxy)-disiloxane, poly(methyl)-siloxanes and poly(methylphenyl)siloxanes, and the like. Other synthetic lubricating oils include liquid esters of phosphorus-containing acids (e.g., tricresyl phosphate, trioctyl phosphate, and diethyl ester of decylphosphonic acid), polymeric tetrahydrofurans, poly-[alpha]-olefins, and the like.

The lubricating oils may be derived from refined, rerefined oils, or mixtures thereof. Unrefined oils are obtained directly from a natural source or synthetic source (e.g., coal, shale, or tar sands bitumen) without further purification or treatment. Examples of unrefined oils include a shale oil obtained directly from a retorting operation, a petroleum oil obtained directly from distillation, or an ester oil obtained directly from an esterification process, each of which is then used without further treatment. Refined oils are similar to the unrefined oils except that refined oils have been treated in one or more purification steps to improve one or more properties. Suitable purification techniques include distillation, hydrotreating, dewaxing, solvent extraction, acid or base extraction, filtration, and percolation, all of which are known to those skilled in the art. Rerefined oils are obtained by treating used oils in processes similar to those used to obtain the refined oils. These rerefined oils are also known as reclaimed or reprocessed oils and are often additionally processed by techniques for removal of spent additives and oil breakdown products.

Once selected the lubricant oil, the incorporation of the nanoobjects of the present invention can be made by any of the routinely techniques, such as simply addition and agitation. The amount of nanoobject to be added can be easily determined by the skilled of the art, and it will depend on the nature and amount of the lubricant oil as well as of its intended use. In general terms, the % by weight of nanoobjects with respect to the total weight of lubricant composition will be in the range comprised from 0.01 to 10.00% by weight, preferably from 0.25 to 2.00%.

Due to the properties of the nanoobjects of the invention, they can also be useful in reducing the wear of a particular composite.

The nanoobjects can also be useful in reducing the wear of a polymeric matrix.

“Composite” is a material made from two or more constituent materials with significantly different physical or chemical properties that, when combined, produce a material with characteristics different from the individual components. The individual components remain separate and distinct within the finished structure. The term “nanocomposite” implies that one of the constituent materials is composed of objects with nanodimensions. The main advantage of the nanocomposites is their vastly improved properties (such as mechanical, physico-chemical, and others) with a relatively small content of the nanomaterial, mainly due to its large surface area.

In one embodiment, the composite comprises a polymeric, matrix, the reinforcing fibre, and a nanoobject of the first aspect of the invention. The amount of nanoobject present in the particle-containing polymer composition and polymer article will vary depending on the end use application. However, typically, the amount of nanoobject of the first aspect of the invention can be comprised from about 0.01 to about 20 wt %, based on the total weight of the composition, preferably, from about 0.2 to about 5 wt %.

The polymeric reinforced matrix comprises a polymericmatrix, and a reinforcing fibre,

The polymeric matrix is a thermosetting or thermoplastic polymer selected from the group consisting of: alkyd resin, epoxy resin, phenolic resin, polyvinyl halides, polyacetal, polyacrylics, polyalkylenes, polyalkenylenes, polyalkynylenes, polyamic acids, polyamides, polyamines, polyanhydrides, polyarylenealkylenes, polyarylenes, polyazomethines, polybenzimidazoles, polybenzothiazoles, polybenzyls, polycarbodiimides, polycarbonates, polycarbones, polycarboranes, polycarbosilanes, polycyanurates, polydienes, polyester-polyurethanes, polyesters, polyetheretherketones, polyether-polyurethanes, polyethers, polyhydrazides, polyimidazoles, polyimides, polyisocyanurates, polyketones, polyolefines, polyoxyalkylenes, polyoxyphenylenes, polyphenyls, polyphosphazenes, polypyrroles, polypyrrones, polyquinolines, polyquinoxalines, polysilanes, polysilazanes, polysiloxanes, polysilsesquioxanes, polysulfides, polysulfonamides, polysulfones, polythiazoles, polythiomethylenes, polythiophenylenes, polyureas, polyurethanes, polyvinyl acetals, polyvinyl butyrals, polyvinyl formals, polyvinyl alkanoates, vinyl polymers, elastomers (such as ACM, AEM, AU, BIIR, BR, CIIR, CR, CSM, ECO, EP, EPDM, EU, FFKM, FKM, FMQ, FPM, HNBR, IR, IIR, NBR, PU, SBR, SEBS, SI, VMQ, XNBR, XSBR, YBPO, YSBR, YXSBR elastomers which are herein referred following ISO 1629 standard) and natural polymers.

The reinforcement fibre can be selected from the group comprising glass fibres, carbon fibres, boron fibres, ceramic fibres like SiC or Al₂O₃, synthetic polymer fibres such as aramid fibres and natural fibres like cellulose fibres.

Once selected the polymer matrix and the fibre, the incorporation of the nanoobjects of the invention can be done following the fabrication techniques mentioned below. In addition to these techniques, there are other processes for fabrication of nanocomposites like solid intercalation, covulcanization, sol-gel method, in-situ formation, and slurry compounding.

In one embodiment, the percentage by weight of nanoobjects of the invention with respect to the total weight of the composite can be comprised from 0.01 to 20%. In another embodiment, the percentage by weight of nanoobjects of the invention with respect to the total weight of the composite is comprised from 0.1 to 7%. In another embodiment, the percentage by weight of nanoobjects of the invention with respect to the total amount of composite is comprised from 0.2 to 5%.

In another embodiment, the composite comprises a polymeric reinforced matrix at a percentage by weight comprised from 0.1 to 90%. In another embodiment, the composite comprises a glass fibre reinforced polyamide matrix at a percentage by weight comprised from 1 to 70%. In another embodiment, the composite comprises a glass fibre reinforced polyamide matrix at a percentage by weight comprised from 5 to 55%.

In another embodiment, the composite comprises a polymeric reinforced matrix at a percentage by weight comprised from 0.1 to 90%. In another embodiment, the composite comprises a glass fibre reinforced polypropylene matrix at a percentage by weight comprised from 1 to 70%. In another embodiment, the composite comprises a glass fibre reinforced polypropylene matrix at a percentage by weight comprised from 5 to 55%.

In-situ polymerization involves the chemical reaction by means of covalent bonds between the reinforcement and the monomer (any molecule of low molecular weight capable of reacting with identical or different molecules of low molecular weight to form a polymer) to address an adequate dispersion and developing strong interfacial adhesion between both of them, essential to enhance composite properties. This method showed good exfoliation of the clay in the polymer matrix. It is efficient in producing nanocomposites especially for thermosetting polymeric matrices.

Methods for obtaining nanocomposites based on the use of solutions, such as solution blending or in combination with any of the methods available, such as those mentioned above could be useful to obtain nanocomposites. Melt processing, sometimes referred to as melt intercalation, or melt blending is the process of compounding the polymeric matrix with the nanoobjects during melting. The technique has been used extensively in the literature to produce nanocomposites.

The nanoobject of the first aspect of the invention may be contacted with a first high molecular weight melt processable polymer. Any melt compounding techniques, known to those skilled in the art may be used. Generally, the nanoobjects, other additives like natural or glass fibres and melt-processable polymer are brought together and then mixed in a blending operation, such as dry blending, that applies shear to the polymer melt to form the particle containing, more typically pigmented, polymer. The melt-processable polymer is usually available in the form of granules, pellets or cubes. Methods for dry blending include shaking in a bag or tumbling in a closed container. Other methods include blending using agitators or paddles. Nanoobject, and melt-processable polymer may be co-fed using screw devices, which mix the treated particle, polymer and melt-processable polymer together before the polymer reaches a molten state. Alternatively, the components may be fed separately into equipment where they may be melt blended, using any methods known in the art, including screw feeders, kneaders, high shear mixers, blending mixers, and the like. Typical methods use Banbury mixers, single and twin screw extruders, and hybrid continuous mixers.

Processing temperatures depend on the polymer and the blending methods used and can be easily determined by those skilled in the art. The intensity of mixing depends on the polymer characteristics. The amount of nanoobject present in the particle-containing polymer composition and polymer article will vary depending on the end use application. However, typically, the amount of nanoobject of the first aspect of the invention is comprised from about 0.01 to about 20 wt %, based on the total weight of the composition, preferably, about 0.2 to about 5 wt %.

Twin-screw extruders are commonly used to mix nanoobjects and melted polymer. Co-rotating twin-screw extruders are available from Brabender. The melt blended polymer mixed with nanoobjects is extruded to form a shaped article. Nanoobjects in accordance with this disclosure are capable of being dispersed throughout the polymer melt. The nanoobjects can be uniformly dispersed throughout the polymer melt.

In one embodiment, the nanocomposite comprises a glass fibre reinforced polyamide and nanoobjects of the first aspect of the invention. In another embodiment, the composite comprises a glass fibre reinforced polyamide matrix at a percentage by weight comprised from 0.1 to 90%. In another embodiment, the composite comprises a glass fibre reinforced polyamide matrix at a percentage by weight comprised from 1 to 70%. In another embodiment, the composite comprises a glass fibre reinforced polyamide matrix at a percentage by weight comprised from 5 to 55%. In another embodiment, the composite comprises a glass fibre reinforced polyamide matrix at a percentage by weight comprised from 0.1 to 90% and nanoobjects of the invention at a percentage by weight with respect to the total weight of the composite comprised from 0.01 to 20%, wherein the sum of the components is up to 100. In another embodiment, the composite comprises a glass fibre reinforced polyamide matrix at a percentage by weight, with respect to the total weight of the composite, comprised from 0.1 to 90% and nanoobjects of the invention at a percentage by weight with respect to the total weight of the composite comprised from 0.1 to 7%. In another embodiment, the composite comprises a glass fibre reinforced polyamide matrix at a percentage by weight, with respect to the total weight of composite, comprised from 0.1 to 90%, and nanoobjects of the invention at a percentage by weight with respect to the total amount of composite comprised from 0.2 to 5%.

In another embodiment, the composite comprises a glass fibre reinforced polyamide matrix at a percentage by weight, with respect to the total weight of the composite, comprised from 1.0 to 70% and nanoobjects of the invention at a percentage by weight with respect to the total weight of the composite comprised from 0.01 to 20%. In another embodiment, the composite comprises a glass fibre reinforced polyamide matrix at a percentage by weight, with respect to the total weight of the composite, comprised from 1.0 to 70% and nanoobjects of the invention at a percentage by weight with respect to the total weight of the composite comprised from 0.1 to 7%. In another embodiment, the composite comprises a glass fibre reinforced polyamide matrix at a percentage by weight, with respect to the total weight of composite, comprised from 1.0 to 70%, and nanoobjects of the invention at a percentage by weight with respect to the total amount of composite comprised from 0.2 to 5%.

In another embodiment, the composite comprises a glass fibre reinforced polyamide matrix at a percentage by weight, with respect to the total weight of the composite, comprised from 5.0 to 55% and nanoobjects of the invention at a percentage by weight with respect to the total weight of the composite comprised from 0.01 to 20%. In another embodiment, the composite comprises a glass fibre reinforced polyamide matrix at a percentage by weight, with respect to the total weight of the composite, comprised from 5.0 to 55% and nanoobjects of the invention at a percentage by weight with respect to the total weight of the composite comprised from 0.1 to 7%. In another embodiment, the composite comprises a glass fibre reinforced polyamide matrix at a percentage by weight, with respect to the total weight of composite, comprised from 5.0 to 55%, and nanoobjects of the invention at a percentage by weight with respect to the total amount of composite comprised from 0.2 to 5%.

In one embodiment, the nanocomposite comprises a glass fibre reinforced polypropylene and nanoobjects of the first aspect of the invention. In another embodiment, the composite comprises a glass fibre reinforced polypropylene matrix at a percentage by weight comprised from 0.1 to 90%. In another embodiment, the composite comprises a glass fibre reinforced polypropylene matrix at a percentage by weight comprised from 1 to 70%. In another embodiment, the composite comprises a glass fibre reinforced polypropylene matrix at a percentage by weight comprised from 5 to 55%. In another embodiment, the composite comprises a glass fibre reinforced polypropylene matrix at a percentage by weight comprised from 0.1 to 90% and nanoobjects of the invention at a percentage by weight with respect to the total weight of the composite comprised from 0.01 to 20%, wherein the sum of the components is up to 100. In another embodiment, the composite comprises a glass fibre reinforced polypropylene matrix at a percentage by weight, with respect to the total weight of the composite, comprised from 0.1 to 90% and nanoobjects of the invention at a percentage by weight with respect to the total weight of the composite comprised from 0.1 to 7%. In another embodiment, the composite comprises a glass fibre reinforced polypropylene matrix at a percentage by weight, with respect to the total weight of composite, comprised from 0.1 to 90%, and nanoobjects of the invention at a percentage by weight with respect to the total amount of composite comprised from 0.2 to 5%.

In another embodiment, the composite comprises a glass fibre reinforced polypropylene matrix at a percentage by weight, with respect to the total weight of the composite, comprised from 1.0 to 70% and nanoobjects of the invention at a percentage by weight with respect to the total weight of the composite comprised from 0.01 to 20%. In another embodiment, the composite comprises a glass fibre reinforced polypropylene matrix at a percentage by weight, with respect to the total weight of the composite, comprised from 1.0 to 70% and nanoobjects of the invention at a percentage by weight with respect to the total weight of the composite comprised from 0.1 to 7%. In another embodiment, the composite comprises a glass fibre reinforced polypropylene matrix at a percentage by weight, with respect to the total weight of composite, comprised from 1.0 to 70%, and nanoobjects of the invention at a percentage by weight with respect to the total amount of composite comprised from 0.2 to 5%.

In another embodiment, the composite comprises a glass fibre reinforced polypropylene matrix at a percentage by weight, with respect to the total weight of the composite, comprised from 5.0 to 55% and nanoobjects of the invention at a percentage by weight with respect to the total weight of the composite comprised from 0.01 to 20%. In another embodiment, the composite comprises a glass fibre reinforced polypropylene matrix at a percentage by weight, with respect to the total weight of the composite, comprised from 5.0 to 55% and nanoobjects of the invention at a percentage by weight with respect to the total weight of the composite comprised from 0.1 to 7%. In another embodiment, the composite comprises a glass fibre reinforced polypropylene matrix at a percentage by weight, with respect to the total weight of composite, comprised from 5.0 to 55%, and nanoobjects of the invention at a percentage by weight with respect to the total amount of composite comprised from 0.2 to 5%.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

Materials

Sodium molybdate (Na₂MoO₄≥98%), thiourea (puriss. p.a., Reag. Ph. Eur., ≥99%), 1,6 hexanediol (HD, 97%), 1,6-hexanedithiol (HDT) (96%), 6-amino-1-hexanol (AHOL) (97%) and Poly(ethylene glycol) mono methyl ether (PEGM, Mw 10000) were purchased from Sigma-Aldrich. Diethylene glycol (DEG, Synthesis grade), diethanolamine (DEA, ≥95%) and ethylene glycol (EG, Synthesis grade) were purchased from Scarlab. 1,2-Propanediol (PG, 99%, extra pure) was purchased from Acros Organics. Poly(ethylene glycol) (PEG, Mw 10000) was purchased from Fluka. All reagents were used as received.

Glass fiber reinforced polypropylene (PP CMP GB366WG NAT) was purchased from BOREALIS and glass fiber reinforced polyamide (Zytel® 80G33HS1L NC010) was purchased from DUPONT.

Experimental Techniques

Fourier Transform Infrared spectroscopy (FTIR) was recorded at room temperature on a Nicolet Avatar 360 spectrophotometer. The spectra were taken with a 4 cm⁻¹ resolution in a wave-number range from 4000 to 400 cm⁻¹.

Thermal gravimetric analysis (TGA) measurements were carried out with a TA-Instrument Q500 TGA using a temperature range from 30 to 300° C. at a heating rate of 10° C.·min⁻ under air.

The micromorphology of the samples was evaluated using a Field Emission Scanning Electron Microscopy (FE-SEM). The measurements were carried out with a Carl Zeiss Ultra Plus field emission scanning electron microscope equipped with an energy dispersive X-ray spectrometer (EDXS).

The micromorphology of the samples was also evaluated using a Transmission Electron Microscopy (TEM). TEM observations were performed on JEOL 2010 F operating with 200 KV accelerating voltage equipped with Energy Dispersive Spectroscopy (EDS). The characterization of nanoobjects was made by deposition of a drop of highly diluted (0.1 mg/mL) nanoobjects dispersion in heptane onto a formvar coated grid, stabilized with evaporated carbon film, FCF300-Cu-25 grid from Electron Microscopy Science. The sizes of pitch, hole and bar are 84, 61, 23 μm, respectively (300 mesh)

A pin-on-disc test equipment (CSM Instrument) was used to carry out tribological tests. In case of oils, a load of 10 N and a speed of 12 cm·s⁻¹ was used in the tests. The friction coefficient was monitored constantly during the tribotests. Ac100Cr6 ball and disk were used in the tribological test. The test duration was 4000 s and was carried out at 120° C. On the other hand, in case of nanocomposites, the temperature of tests was 25° C., the load was 5N (nanocomposites based on polypropylene) or 10 N (nanocomposites based on polyamide), and a speed of 12 cm·s⁻¹. A Ac100Cr6 ball and nanocomposites disk were used in the tribological test. The test duration was 3750 s.

The wear of nanocomposite test specimens was measured using an Ambios Technology XP-1 profilometer. The speed and the length of measure was 2 mm·s-1 and 7 mm, respectively. The stylus tracking force was 2 mg. The wear was calculated by measuring the difference between the surface of test specimen and the lowest point of wearing groove.

### Example 1: Synthesis of MoS₂ 0D Nanoobjects Functionalized with DEG

A total amount of 0.05 mmol of sodium molybdate and 0.28 mmol of thiourea were stirred in 7.68 mL diethylene glycol (DEG) under air atmosphere at 220° C. for 180 min.

After that, the reactor was quenched to room temperature and nanoobjects were isolated and purified. To remove the excess of reactants, solvent and co-products, the samples were washed by centrifugation: two times with ethanol, another two times with pure water and finally were washed one time with ethanol. Finally, the nanoobjects were dried at room temperature.

After purification, synthesized OD nanoobjects were characterized by FE-SEM, TEM, FTIR, and TGA.

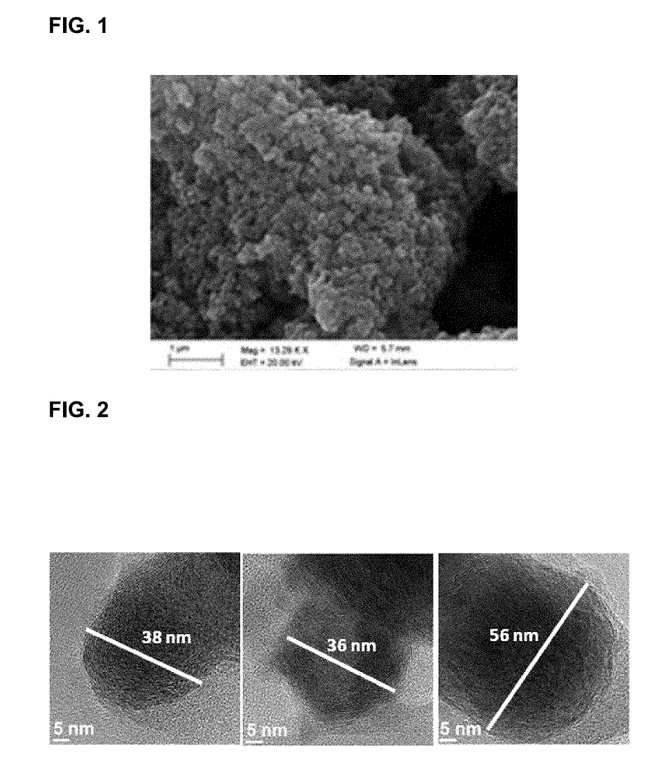

FIG. 1 shows FE-SEM micrographs of agglomerates of primary nanoobjects synthesized in DEG and quenched. FIG. 2 confirms the sphere-like primary morphology of the MoS₂ nanoobjects. As can be observed in these TEM micrographs, they have spherical shape and almost uniform object size of 40 nm.

FTIR spectra of the synthesized nanoobjects shows a band around 3400 cm⁻¹ corresponding to the stretching band of the O—H bond, and another one at around 1099 cm⁻¹ corresponding to the stretching band of the C—O bond. The presence of these bands confirms the functionalization of the nanoobject with DEG.

The mass loss (%) of DEG was measured by TGA. The organic content of the OD nanoobjects of this example 1 was 46% (weight)

### Example 2: Synthesis of MoS₂ 2D Nanoobjects Functionalized with DEG

A total amount of 0.05 mmol of sodium molybdate and 0.28 mmol of thiourea were stirred in 7.68 mL diethylene glycol (DEG) under air atmosphere at 220° C. for 180 min.

After that, the reactor was cooled at 15° C./h to room temperature and nanoobjects were isolated and purified. To remove the excess of reactants, solvent and co-products, the samples were washed by centrifugation: two times with ethanol, another two times with pure water and finally were washed one time with ethanol. Finally, the nanoobjects were dried at room temperature.

After purification, synthesized 2D nanoobjects were characterized by FE-SEM, TEM, FTIR, and TGA.

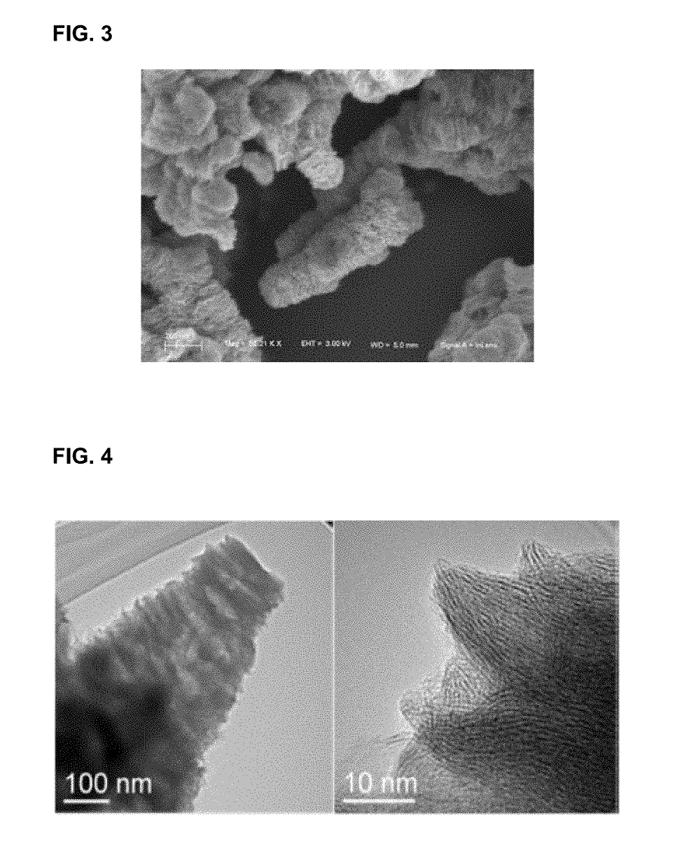

FIG. 3 shows FE-SEM micrograph of agglomerates of primary nanoobjects synthesized in DEG and cooled at 15° C./h. It can be observed the nanosheets agglomerated in cylinders.

FIG. 4 shows more clearly the primary morphology of the MoS₂ nanoobjects of this example. As it can be observed in these TEM micrographs, they have sheet shape and almost uniform sizes, with an average object size of 0.6 nm.

FTIR spectra of the synthesized nanoobjects shows a band around 3400 cm⁻¹ corresponding to the stretching band of the O—H bond, and another one at around 1099 cm⁻¹ corresponding to the stretching band of the C—O bond. The presence of these bands confirms the functionalization of the nanoobject with the DEG.

The mass loss (%) of DEG was measured by TGA. The organic content of the OD nanoobjects of this example 2 was 52% (weight).

### Example 3: Synthesis of MoS₂ 0D Nanoobjects Functionalized with Different Molecules

Following the procedure of Example 1, different nanoobjects were synthesized using molecules of formula (I) other than DEG. The molecules used were: ethylene glycol (EG), 1,2-propylen glycol (PG), polyethyleneglycol (Molecular weight 10000) (PEG10000), Poly(ethylene glycol) monomethyl ether (PEGM 10000) diethanolamine (DEA), 1,6-hexanediol (HD), 1,6-hexanedithiol (HDT) and 6-amino-1-hexanol (AHOL).

It was confirmed by SEM that the structure of the resulting nanoobjects was substantially identical to that of Example 1, concluding that the morphology for all the cases was OD.

Table 1 collects the results of organic content of the nanoobjects measured by TGA.

TABLE 1
Organic content of the nanoobjects functionalized with
different molecules

| | | Organic content |
|---|---|---|
| Sample | Molecule | (wt %) |
| Ex. 3.1. = 0D-EG | ethylene glycol | 52 |
| Ex. 3.2. = 0D-PG | 1,2-propylen glycol | 40 |
| Ex. 3.3. = 0D-DEA | diethanolamine | 44 |
| Ex. 3.4. = 0D-HD | 1,6-hexanediol | 43 |
| Ex. 3.5. = 0D-PEG10000 | polyethyleneglycol | 94 |
| Ex. 3.6. = 0D-PEGM10000 | Polyethyleneglycol | 39 |
| | monomethyl ether | |
| Ex. 3.7. = 0D-AHOL | 6-amino-1-hexanol | 36 |
| Ex. 3.8. = 0D-HDT (for | 1,6-hexanedithiol | 51 |
| comparative purposes) | | |

### Example 4: Preparation of Oil with MoS₂ Nanoobjects

Castrol Edge 5W30 oil with MoS₂ nanoobjects were prepared by ball milling. 34.65 g of final oil and 0.35 g of MoS₂ nanoobjects synthesized in any of the previous examples 1 to 3 were incorporated to the container and stirred for 3 h. Each 30 min the stir was stopped for 5 min in order to avoid the warning of samples. The mixture was removed from container and used to tribological test.

It was checked that the nanoobjects were appropriately dispersed in the lubricant composition and that this dispersability was maintained by about at least 20 days.

### Example 5: The Nanoobjects of Invention as Coefficient Friction Reducing Agent when Formulated in an Oil

Tribological tests were carried out with the final formulated oils prepared in Example 4. The results of friction coefficient after 4000 seconds are showed in Table 2:

TABLE 2
Friction coefficient of neat oil and oil with
different kind of difunctional molecules

| | | % Friction |
|---|---|---|
| | μ after | coefficient |
| Sample | 4000 seg | reduction |
| OIL | 0.1786 | — |
| OIL + nanoobject Ex. 1 | 0.1038 | 41.88 |
| OIL + nanoobject Ex. 2 | 0.0992 | 44.44 |
| OIL + nanoobject Ex. 3.1 | 0.0867 | 51.46 |
| OIL + nanoobject Ex. 3.2. | 0.0702 | 60.66 |
| OIL + nanoobject Ex. 3.3. | 0.0924 | 48.29 |
| OIL + nanoobject Ex. 3.4. | 0.0994 | 44.37 |
| OIL + nanoobject Ex. 3.5. | 0.1079 | 39.61 |
| OIL + nanoobject Ex. 3.6. | 0.1040 | 41.77 |
| OIL + nanoobject Ex. 3.7. | 0.1044 | 41.54 |
| OIL + nanoobject Ex. 3.8. (for | 0.1246 | 30.24 |
| comparative purposes) | | |

As it can be seen, the friction coefficient reduction is between 40 and 60% approximately when the nanoobject is functionalized with molecules having at least one —OH group (Ex. 3.1 to Ex. 3.7). However, when the nanoobject is functionalized with molecules having —SH groups (Ex. 3.8), the friction coefficient reduction is significantly lower (around 30%). It is noted that the friction coefficient of the sample with nanoobjects functionalized with —SH groups is very similar to that with non-functionalized nanoobjects (see Table 3).

In order to study the influence of functionalization of the surface of nanoobjects with molecules of formula (I), several tribological tests were carried out in final formulated oil with non-functionalized nanoobjects. The non-functionalized nanoobjects were obtained submitting the funtionalized nanoobjects to a thermal treatment at 300° C. for 2 min. Once obtained the non-functionalized nanoobjects, the friction coefficient was determined and compared with the coefficient reduction value corresponding to the functionalized nanoobject. This assay was performed with the nanoobjects of Examples 1 and 2 and the results are summarized in Table 3 below. As it can be seen, when the functionalizing molecule is removed, the friction coefficient increases, either for the nanoparticle (Example 1) or the nanosheet Example 2).

It can be concluded that an improvement in the performance of the nanoobjects is achieved when they are functionalized with molecules of formula (I) of the invention, achieving a reduction higher than a 10% in the friction coefficient

TABLE 3
Oil friction coefficient and % friction coefficient increase using
non-functionalized MoS₂ nanoobjects compared to the
corresponding functionalized nanoobjects

| | | % Friction |
|---|---|---|
| | | coefficient |
| Sample | μ | increase |
| OIL + nanoobject | 0.10384 | — |
| Ex. 1 | | |
| OIL + non- | 0.12455 | 16.66 |
| functionalized | | |
| nanoobject of Ex. 1 | | |
| OIL + nanoobject | 0.09927 | — |
| Ex. 2 | | |
| OIL + non- | 0.11488 | 13.58 |
| functionalized | | |
| nanoobject Ex. 2 | | |

### Example 6: Preparation of Nanocomposites Based on Glass Fibre Reinforced Polyamide

Nanocomposites were prepared by melt blending using a Brabender Twin Screw Extruder R DSE 20/40. MoS₂ nanoobjects of Example 1 were melt blended with polyamide 6,6 (“PA”) reinforced with 33 wt % glass fibre (“GF”) (Zytel® 80G33HS1L NC010) on co-rotating twin screw extruder with a flat temperature profile at 260° C. and screw speed of 100 rpm and the extrudate was pelletized. The polyamide 6,6 reinforced with 33 wt % glass fibre pellets containing 1 and 2.6 wt % of MoS₂ nanoobjects were fed separately.

For comparison purposes, composites containing only polyamide 6,6 reinforced with 33% of glass fibre were used. Table 4 below collects the wearing of the three systems.

TABLE 4
Composites wear and % reduction of wear using functionalized
MoS₂ nanoobjects (composite with MoS₂ nanoobjects
vs. composite without MoS₂ nanoobjects)

| Sample | wear (μm) | % wear reduction |
|---|---|---|
| PA33 wt % GF | 11.90 | — |
| PA33 wt % GF + 1 wt % Nanoobject | 9.69 | 18.6 |
| Ex. 1 | | |
| PA33 wt % GF + 2.6 wt % Nanoobject | 4.92 | 58.6 |
| Ex. 1 | | |

The results allow concluding that nanoobjects functionalized with molecules of formula (I) have a huge influence in the reduction of the wear of glass fibre reinforced PA polymeric matrices.

### Example 7: Preparation of Nanocomposites Based on Glass Fibre Reinforced Polypropylene

Nanocomposites were prepared by melt blending using a Brabender Twin Screw Extruder R DSE 20/40. MoS₂ nanoobjects of Example 1 were melt blended with polypropylene (“PP”) reinforced with 30 wt % glass fibre (“GF”) (PP CMP GB366WG NAT) on co-rotating twin screw extruder with a flat temperature profile at 260° C. and screw speed of 100 rpm and the extrudate was pelleted. The polypropylene reinforced with 30 wt % glass fibre pellets containing 0.5, 1 and 2.6 wt % of MoS₂ nanoobjects were fed separately.

For comparison purposes, composites containing only polypropylene reinforced with 30% of glass fibre were used. Table 5 below collects the wear of the four systems.

TABLE 5
Composites wear and % reduction of wear using functionalized
MoS₂ nanoobjects (composite with MoS₂ nanoobjects
vs. composite without MoS₂ nanoobjects)

| Sample | wear (μm) | % wear reduction |
|---|---|---|
| PP30 wt % GF | 11.19 | — |
| PP30 wt % GF + 0.5 wt % Nanoobject | 8.37 | 25.2 |
| Ex. 1 | | |
| PP30 wt % GF + 1 wt % Nanoobject | 8.06 | 28.0 |
| Ex. 1 | | |
| PP30 wt % GF + 2.6 wt % Nanoobject | 6.48 | 42.1 |
| Ex. 1 | | |

The results allow concluding that nanoobjects functionalized with molecules of formula (I) have a huge influence in the reduction of the wear of glass fibre reinforced PP polymeric matrices.

### REFERENCES CITED IN THE APPLICATION

US 20050065044, and
Parenago O. P. et al., “Synthesis and applications of inorganic nanoobjects as lubricant components—a review”, 2004, Journal of Nanoparticle Research, vol. 6, pages 273-284.

1. A Molybdenum or Tungsten chalcogenide nanoobject having: (a) an object size comprised from 0.1 to 500 nm, and (b) from 1 to 99% by weight of molecules of formula (I) with respect to the total weight of the nanoobject
A-X-B (I)
wherein
A is —OH;
X is a biradical selected from the group consisting of: (C₁-C₂₀)alkyl; (C₁-C₂₀)alkyl substituted with one or more (C₁-C₅)alkyl, —OH, halogen, phenyl, phenyl substituted with one or more (C₁-C₄)alkyl radicals, phenyl substituted with one or more halogen radicals, benzyl, benzyl substituted with one or more (C₁-C₄)alkyl radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), — S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂), and —B(OR₁)(R₂); a 2 to 20-member heteroalkyl; a 2 to 20-member heteroalkyl substituted with one or more —OH, halogen, phenyl, phenyl substituted with one or more (C₁-C₄)alkyl radicals, phenyl substituted with one or more halogen radicals, benzyl, benzyl substituted with one or more (C₁-C₄)alkyl radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), — S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂) and —B(OR₁)(R₂); and a homopolymer or copolymer comprising a polymeric chain selected from the group consisting of: alkyd resin, epoxy resin, phenolic resin, polyvinyl halides, polyacetal, polyacrylics, polyalkylenes, polyalkenylenes, polyalkynylenes, polyamic acids, polyamides, polyamines, polyanhydrides, polyarylenealkylenes, polyarylenes, polyazomethines, polybenzimidazoles, polybenzothiazoles, polybenzyls, polycarbodiimides, polycarbonates, polycarbones, polycarboranes, polycarbosilanes, polycyanurates, polydienes, polyester-polyurethanes, polyesters, polyetheretherketones, polyether-polyurethanes, polyethers, polyhydrazides, polyimidazoles, polyimides, polyisocyanurates, polyketones, polyolefines, polyoxyalkylenes, polyoxyphenylenes, polyphenyls, polyphosphazenes, polypyrroles, polypyrrones, polyquinolines, polyquinoxalines, polysilanes, polysilazanes, polysiloxanes, polysilsesquioxanes, polysulfides, polysulfonamides, polysulfones, polythiazoles, polythiomethylenes, polythiophenylenes, polyureas, polyurethanes, polyvinyl acetals, polyvinyl butyrals, polyvinyl formals, polyvinyl alkanoates, vinyl polymers, and a natural polymer;
B is a radical selected from the group consisting of: H, —NH₂, (C₁-C₄)alkyl, OH, halogen, phenyl substituted with one or more halogen radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), —S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂) and —B(OR₁)(R₂);
provided that:
B is —H or (C₁-C₄) alkyl when X is a homopolymer, copolymer, a 2 to 20-member heteroalkyl or a 2 to 20-member heteroalkyl substituted as defined above; and
when B is —NH₂, then X is a biradical selected from the group consisting of: (C₁-C₂₀)alkyl; (C₁-C₂₀)alkyl substituted with one or more (C₁-C₅)alkyl, —OH, halogen, phenyl, phenyl substituted with one or more (C₁-C₄)alkyl radicals, phenyl substituted with one or more halogen radicals, benzyl, benzyl substituted with one or more (C₁-C₄)alkyl radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), — S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂), and —B(OR₁)(R₂); a 2 to 20-member heteroalkyl; a 2 to 20-member heteroalkyl substituted with one or more —OH, halogen, phenyl, phenyl substituted with one or more (C₁-C₄)alkyl radicals, phenyl substituted with one or more halogen radicals, benzyl, benzyl substituted with one or more (C₁-C₄)alkyl radicals, benzyl substituted with one or more halogen radicals, —C(═O)R₃, —C(═O)(R₇), —OC(═O)(O)R₃, —C(═O)(O⁻), —C(═O)(O)R₃, —OR₃, —CH(OR₃)(OR₄), —C(OR₃)(OR₄)(R₅), —C(OR₃)(OR₄)(OR₅), —C(OR₃)(OR₄)(OR₅)(OR₆), —NR₁R₂, —N⁺R₁R₂R₃, —C(═NR₁)(R₂), —C(═O)(NR₁R₂), —N(C(═O)(R₁)) (C(═O)(R₂))(R₃), —O(CN), —NC(═O), —ONO₂, —CN, —NC, —ON(═O), —NO₂, —NO, —C₅H₄N, —SR₁, —SSR₁, —S(═O)(R₁), —S(═O)(═O)(R₁), —S(═O)(OH), —S(═O)(═O)(OH), —SCN, —NCS, —C(═S)(R₁), —PR₁R₂, —P(═O)(OH)₂, —OP(═O)(OH)₂, —OP(═O)(OR₁)(OR₂), —B(OH), —B(OR₁)(OR₂) and —B(OR₁)(R₂);
R₁, R₂, R₃, R₄, R₅, and R₆ are radicals independently selected from the group consisting of —H, (C₁-C₂₀)alkyl, (C₅-C₁₂)aryl(C₁-C₂₀)alkyl and (C₅-C₁₂)aryl;
R₇ is halogen;
2 to 20-member heteroalkyl represents a known non-polymeric C-heteroalkyl radical consisting of from 2 to 20 members where at least one of the members is O, S, or NH, and the remaining members are selected from CH, C(═O), and CH₂; and
(C₅-C₁₂)aryl represents a ring system from 5 to 12 carbon atoms, the system comprising from 1 to 2 rings, where each one of the rings forming the ring system: is saturated, partially unsaturated, or aromatic; and is isolated, partially fused or totally fused.

2. The nanoobject of claim 1, wherein the chalcogenide nanoobject is a Molybdenum chalcogenide of sulfide, selenide or telluride.

3. The nanoobject of claim 2, wherein the metal chalcogenide nanoobject is MoS₂.

4. The nanoobject of claim 1, wherein A represents —OH.

5. The nanoobject of claim 1, wherein X is selected from the group consisting of (C₁-C₁₀)alkyl, a 2- to 10-member heteroalkyl, and a homopolymer or copolymer comprising a polyether chain.

6. The nanoobject of claim 1, wherein B is OH, NH₂, (C₁-C₄)alkyl, or H.

7. The nanoobject of claim 1, wherein B is OH or H.

8. The nanoobject of claim 1, wherein the molecule of formula (I) is selected from the group consisting of propylene glycol, ethylene glycol, diethylene glycol, polyethylene glycol, diethanolamine, 1,6-hexanediol, polyethyleneglycol monomethyl ether, and 6-amino-1-hexanol.

9. The nanoobject of claim 1, wherein the molecule of formula (I) is selected from the group consisting of propylene glycol, ethylene glycol, diethylene glycol, polyethylene glycol, diethanolamine, and 1,6-hexanediol.

10. A process for preparing a nanoobject according to claim 1, comprising the step of reacting a Molybdenum or Tungsten source with a source of S, Se or Te, and molecules of formula (I) as defined in claim 1.

11. The process of claim 10 wherein the Molybdate or Tungstate salt reacts with an urea compound selected from the group consisting of: NR₈R₉—C(═S)—NR₁₀R₁₁, NR₈R₉—C(═Se)—NR₁₀R₁₁, and NR₈R₉—C(═Te)—NR₁₀R₁₁ wherein R₈ to R₁₁ are independently selected from of H, (C₁-C₂₀)alkyl, (C₅-C₁₂)aryl(C₁-C₂₀)alkyl and (C₅-C₁₂)aryl, the term “(C₅-C₁₂)aryl” being as defined in claim 1.

12. The process of claim 10, which comprises reacting a Molybdate salt with NH₂—C(═S)—NH₂.

13. A method for reducing the friction coefficient of a material which comprises the step of adding a nanoobject as defined in claim 1 into said material.

14. The method of claim 13, wherein the material is a lubricant oil.

15. An article comprising the nanoobject as defined in claim 1.

16. A nanocomposite comprising the nanoobject as defined in claim 1.

17. A lubricant oil comprising the nanoobject as defined in claim 1.

18. The nanoobject of claim 1, which is a MoS₂ nanoobject; A represents —OH; B represents OH, NH₂, (C₁-C₄)alkyl, or H; and X is selected from the group consisting of (C₁-C₁₀)alkyl, a 2- to 10-member heteroalkyl, and a homopolymer or copolymer comprising a polyether chain.

19. The nanoobject of claim 1, which is a MoS₂ nanoobject, and the molecule of formula (I) is selected from the group consisting of propylene glycol, ethylene glycol, diethylene glycol, polyethylene glycol, diethanolamine, 1,6-hexanediol, polyethyleneglycol monomethyl ether, and 6-amino-1-hexanol.

20. The article as defined in claim 15, wherein the nanoobject is a MoS₂ nanoobject; A represents —OH; B represents OH, NH₂, (C₁-C₄)alkyl, or H; and X is selected from the group consisting of (C₁-C₁₀)alkyl, a 2- to 10-member heteroalkyl, and a homopolymer or copolymer comprising a polyether chain; or
wherein the nanoobject is a MoS₂ nanoobject, and the molecule of formula (I) is selected from the group consisting of propylene glycol, ethylene glycol, diethylene glycol, polyethylene glycol, diethanolamine, 1,6-hexanediol, polyethyleneglycol monomethyl ether, and 6-amino-1-hexanol.

21. The nanocomposite as defined in claim 16, wherein the nanoobject is a MoS₂ nanoobject; A represents —OH; B represents OH, NH₂, (C₁-C₄)alkyl, or H; and X is selected from the group consisting of (C₁-C₁₀)alkyl, a 2- to 10-member heteroalkyl, and a homopolymer or copolymer comprising a polyether chain; or
wherein the nanoobject is a MoS₂ nanoobject, and the molecule of formula (I) is selected from the group consisting of propylene glycol, ethylene glycol, diethylene glycol, polyethylene glycol, diethanolamine, 1,6-hexanediol, polyethyleneglycol monomethyl ether, and 6-amino-1-hexanol.

22. The lubricant oil as defined in claim 17, wherein the nanoobject is wherein the nanoobject is a MoS₂ nanoobject; A represents —OH; B represents OH, NH₂, (C₁-C₄)alkyl, or H; and X is selected from the group consisting of (C₁-C₁₀)alkyl, a 2- to 10-member heteroalkyl, and a homopolymer or copolymer comprising a polyether chain; or
wherein the nanoobject is a MoS₂ nanoobject, and the molecule of formula (I) is selected from the group consisting of propylene glycol, ethylene glycol, diethylene glycol, polyethylene glycol, diethanolamine, 1,6-hexanediol, polyethyleneglycol monomethyl ether, and 6-amino-1-hexanol.
